SEQUENCE LISTING (136 sequences)

SEQ ID NO: 1 - DNA, 20 bp, synthetic construct
  source 1..20: mol_type = other DNA
gttcgtttagagaacagatc

SEQ ID NO: 2 - DNA, 21 bp, synthetic construct
  source 1..21: mol_type = other DNA
taaagttcgtttagagaacag

SEQ ID NO: 3 - DNA, 15 bp, synthetic construct
  source 1..15: mol_type = other DNA
agccctgtatacgac

SEQ ID NO: 4 - DNA, 19 bp, synthetic construct
  source 1..19: mol_type = other DNA
gtaggtaaaaacctaatat

SEQ ID NO: 5 - DNA, 20 bp, synthetic construct
  source 1..20: mol_type = other DNA
cgtttagagaacagatctac

SEQ ID NO: 6 - DNA, 20 bp, synthetic construct
  source 1..20: mol_type = other DNA
cattgtagatgtcaaaagcc

SEQ ID NO: 7 - DNA, 21 bp, synthetic construct
  source 1..21: mol_type = other DNA
ctccctcatggtggcagttga

SEQ ID NO: 8 - DNA, 18 bp, synthetic construct
  source 1..18: mol_type = other DNA
cggcatgtctattttgta

SEQ ID NO: 9 - DNA, 18 bp, synthetic construct
  source 1..18: mol_type = other DNA
tttgttcctggatgactc

SEQ ID NO: 10 - DNA, 18 bp, synthetic construct
  source 1..18: mol_type = other DNA
gtttgttcctggatgact

SEQ ID NO: 11 - DNA, 18 bp, synthetic construct
  source 1..18: mol_type = other DNA
agtttgttcctggatgac

SEQ ID NO: 12 - DNA, 18 bp, synthetic construct
  source 1..18: mol_type = other DNA
cagtttgttcctggatga

SEQ ID NO: 13 - DNA, 18 bp, synthetic construct
  source 1..18: mol_type = other DNA
tcagtttgttcctggatg

SEQ ID NO: 14 - DNA, 18 bp, synthetic construct
  source 1..18: mol_type = other DNA
ctcagtttgttcctggat

SEQ ID NO: 15 - DNA, 18 bp, synthetic construct
  source 1..18: mol_type = other DNA
tgtgtgtgtgtgcgtgtg

SEQ ID NO: 16 - DNA, 18 bp, synthetic construct
  source 1..18: mol_type = other DNA
gtgtgtgtgtgtgcgtgt

SEQ ID NO: 17 - DNA, 18 bp, synthetic construct
  source 1..18: mol_type = other DNA
tgtgtgtgtgtgtgcgtg

SEQ ID NO: 18 - DNA, 18 bp, synthetic construct
  source 1..18: mol_type = other DNA
gtgtgtgtgtgtgtgcgt

SEQ ID NO: 19 - DNA, 18 bp, synthetic construct
  source 1..18: mol_type = other DNA
tgtgtgtgtgtgtgtgcg

SEQ ID NO: 20 - DNA, 18 bp, synthetic construct
  source 1..18: mol_type = other DNA
gtgtgtgtgtgtgtgtgc

SEQ ID NO: 21 - DNA, 18 bp, synthetic construct
  source 1..18: mol_type = other DNA
tgtgtgtgtgtgtgtgtg

SEQ ID NO: 22 - DNA, 18 bp, synthetic construct
  source 1..18: mol_type = other DNA
gtgtgtgtgtgtgtgtgt

SEQ ID NO: 23 - DNA, 18 bp, synthetic construct
  source 1..18: mol_type = other DNA
tgtgtgtgtgtgtgtgtg

SEQ ID NO: 24 - DNA, 18 bp, synthetic construct
  source 1..18: mol_type = other DNA
gtgtgtgtgtgtgtgtgt

SEQ ID NO: 25 - DNA, 18 bp, synthetic construct
  source 1..18: mol_type = other DNA
tgtgtgtgtgtgtgtgtg

SEQ ID NO: 26 - DNA, 18 bp, synthetic construct
  source 1..18: mol_type = other DNA
gtgtgtgtgtgtgtgtgt

SEQ ID NO: 27 - DNA, 18 bp, synthetic construct
  source 1..18: mol_type = other DNA
tgtgtgtgtgtgtgtgtg

SEQ ID NO: 28 - DNA, 18 bp, synthetic construct
  source 1..18: mol_type = other DNA
gtgtgtgtgtgtgtgtgt

SEQ ID NO: 29 - DNA, 18 bp, synthetic construct
  source 1..18: mol_type = other DNA
cgtgtgtgtgtgtgtgtg

SEQ ID NO: 30 - DNA, 18 bp, synthetic construct
  source 1..18: mol_type = other DNA
gcgtgtgtgtgtgtgtgt

SEQ ID NO: 31 - DNA, 18 bp, synthetic construct
  source 1..18: mol_type = other DNA
tgcgtgtgtgtgtgtgtg

SEQ ID NO: 32 - DNA, 18 bp, synthetic construct
  source 1..18: mol_type = other DNA
gtgcgtgtgtgtgtgtgt

SEQ ID NO: 33 - DNA, 18 bp, synthetic construct
  source 1..18: mol_type = other DNA
agtggcggatctgaactc

SEQ ID NO: 34 - DNA, 18 bp, synthetic construct
  source 1..18: mol_type = other DNA
gagtggcggatctgaact

SEQ ID NO: 35 - DNA, 18 bp, synthetic construct
  source 1..18: mol_type = other DNA
gagtgtgctgcaggtaga

SEQ ID NO: 36 - DNA, 18 bp, synthetic construct
  source 1..18: mol_type = other DNA
tcgagtgtgctgcaggta

SEQ ID NO: 37 - DNA, 18 bp, synthetic construct
  source 1..18: mol_type = other DNA
atcgagtgtgctgcaggt

SEQ ID NO: 38 - DNA, 18 bp, synthetic construct
  source 1..18: mol_type = other DNA
gtgctgttgtagatggaa

SEQ ID NO: 39 - DNA, 18 bp, synthetic construct
  source 1..18: mol_type = other DNA
ggtgctgttgtagatgga

SEQ ID NO: 40 - DNA, 18 bp, synthetic construct
  source 1..18: mol_type = other DNA
tggtgctgttgtagatgg

SEQ ID NO: 41 - DNA, 18 bp, synthetic construct
  source 1..18: mol_type = other DNA
ctggtgctgttgtagatg

SEQ ID NO: 42 - DNA, 18 bp, synthetic construct
  source 1..18: mol_type = other DNA
cctggtgctgttgtagat

SEQ ID NO: 43 - DNA, 18 bp, synthetic construct
  source 1..18: mol_type = other DNA
ccctggtgctgttgtaga

SEQ ID NO: 44 - DNA, 18 bp, synthetic construct
  source 1..18: mol_type = other DNA
tccctggtgctgttgtag

SEQ ID NO: 45 - DNA, 18 bp, synthetic construct
  source 1..18: mol_type = other DNA
gtccctggtgctgttgta

SEQ ID NO: 46 - DNA, 18 bp, synthetic construct
  source 1..18: mol_type = other DNA
agtccctggtgctgttgt

SEQ ID NO: 47 - DNA, 18 bp, synthetic construct
  source 1..18: mol_type = other DNA
ctgggttggagatgttaa

SEQ ID NO: 48 - DNA, 18 bp, synthetic construct
  source 1..18: mol_type = other DNA
gctgggttggagatgtta

SEQ ID NO: 49 - DNA, 18 bp, synthetic construct
  source 1..18: mol_type = other DNA
cgctgggttggagatgtt

SEQ ID NO: 50 - DNA, 18 bp, synthetic construct
  source 1..18: mol_type = other DNA
tagcgctgggttggagat

SEQ ID NO: 51 - DNA, 18 bp, synthetic construct
  source 1..18: mol_type = other DNA
atgtagcgctgggttgga

SEQ ID NO: 52 - DNA, 18 bp, synthetic construct
  source 1..18: mol_type = other DNA
agccattcgccttctgct

SEQ ID NO: 53 - DNA, 18 bp, synthetic construct
  source 1..18: mol_type = other DNA
gtctttatggtgaagcca

SEQ ID NO: 54 - DNA, 18 bp, synthetic construct
  source 1..18: mol_type = other DNA
tgtctttatggtgaagcc

SEQ ID NO: 55 - DNA, 18 bp, synthetic construct
  source 1..18: mol_type = other DNA
ctgtctttatggtgaagc

SEQ ID NO: 56 - DNA, 18 bp, synthetic construct
  source 1..18: mol_type = other DNA
cctgtctttatggtgaag

SEQ ID NO: 57 - DNA, 18 bp, synthetic construct
  source 1..18: mol_type = other DNA
gttcctgtctttatggtg

SEQ ID NO: 58 - DNA, 18 bp, synthetic construct
  source 1..18: mol_type = other DNA
ggttcctgtctttatggt

SEQ ID NO: 59 - DNA, 18 bp, synthetic construct
  source 1..18: mol_type = other DNA
aggttcctgtctttatgg

SEQ ID NO: 60 - DNA, 18 bp, synthetic construct
  source 1..18: mol_type = other DNA
caggttcctgtctttatg

SEQ ID NO: 61 - DNA, 18 bp, synthetic construct
  source 1..18: mol_type = other DNA
ccaggttcctgtctttat

SEQ ID NO: 62 - DNA, 18 bp, synthetic construct
  source 1..18: mol_type = other DNA
ggtcttcccactgttttt

SEQ ID NO: 63 - DNA, 18 bp, synthetic construct
  source 1..18: mol_type = other DNA
aggagatgtggggtcttc

SEQ ID NO: 64 - DNA, 18 bp, synthetic construct
  source 1..18: mol_type = other DNA
caggagatgtggggtctt

SEQ ID NO: 65 - DNA, 18 bp, synthetic construct
  source 1..18: mol_type = other DNA
ggttggtctgttgtgact

SEQ ID NO: 66 - DNA, 18 bp, synthetic construct
  source 1..18: mol_type = other DNA
cggttggtctgttgtgac

SEQ ID NO: 67 - DNA, 18 bp, synthetic construct
  source 1..18: mol_type = other DNA
ccggttggtctgttgtga

SEQ ID NO: 68 - DNA, 18 bp, synthetic construct
  source 1..18: mol_type = other DNA
gagaatggttagaggttc

SEQ ID NO: 69 - DNA, 18 bp, synthetic construct
  source 1..18: mol_type = other DNA
gtagagaatggttagagg

SEQ ID NO: 70 - DNA, 18 bp, synthetic construct
  source 1..18: mol_type = other DNA
ggtgttttgccaatgtag

SEQ ID NO: 71 - DNA, 18 bp, synthetic construct
  source 1..18: mol_type = other DNA
gggtgttttgccaatgta

SEQ ID NO: 72 - DNA, 18 bp, synthetic construct
  source 1..18: mol_type = other DNA
tgggtgttttgccaatgt

SEQ ID NO: 73 - DNA, 18 bp, synthetic construct
  source 1..18: mol_type = other DNA
catcatcgttgtcgtcgt

SEQ ID NO: 74 - DNA, 18 bp, synthetic construct
  source 1..18: mol_type = other DNA
gaacggtacgtacagcaa

SEQ ID NO: 75 - DNA, 18 bp, synthetic construct
  source 1..18: mol_type = other DNA
ggaacggtacgtacagca

SEQ ID NO: 76 - DNA, 18 bp, synthetic construct
  source 1..18: mol_type = other DNA
aggaacggtacgtacagc

SEQ ID NO: 77 - DNA, 18 bp, synthetic construct
  source 1..18: mol_type = other DNA
taggaacggtacgtacag

SEQ ID NO: 78 - DNA, 18 bp, synthetic construct
  source 1..18: mol_type = other DNA
ataggaacggtacgtaca

SEQ ID NO: 79 - DNA, 18 bp, synthetic construct
  source 1..18: mol_type = other DNA
gataggaacggtacgtac

SEQ ID NO: 80 - DNA, 18 bp, synthetic construct
  source 1..18: mol_type = other DNA
ggataggaacggtacgta

SEQ ID NO: 81 - DNA, 18 bp, synthetic construct
  source 1..18: mol_type = other DNA
gggataggaacggtacgt

SEQ ID NO: 82 - DNA, 18 bp, synthetic construct
  source 1..18: mol_type = other DNA
gggtgcctattgcatagc

SEQ ID NO: 83 - DNA, 18 bp, synthetic construct
  source 1..18: mol_type = other DNA
agggtgcctattgcatag

SEQ ID NO: 84 - DNA, 18 bp, synthetic construct
  source 1..18: mol_type = other DNA
aagggtgcctattgcata

SEQ ID NO: 85 - DNA, 18 bp, synthetic construct
  source 1..18: mol_type = other DNA
gaagggtgcctattgcat

SEQ ID NO: 86 - DNA, 18 bp, synthetic construct
  source 1..18: mol_type = other DNA
ggaagggtgcctattgca

SEQ ID NO: 87 - DNA, 18 bp, synthetic construct
  source 1..18: mol_type = other DNA
tgggaagggtgcctattg

SEQ ID NO: 88 - DNA, 18 bp, synthetic construct
  source 1..18: mol_type = other DNA
atgggaagggtgcctatt

SEQ ID NO: 89 - DNA, 18 bp, synthetic construct
  source 1..18: mol_type = other DNA
aatgggaagggtgcctat

SEQ ID NO: 90 - DNA, 18 bp, synthetic construct
  source 1..18: mol_type = other DNA
gaatgggaagggtgccta

SEQ ID NO: 91 - DNA, 18 bp, synthetic construct
  source 1..18: mol_type = other DNA
agaatgggaagggtgcct

SEQ ID NO: 92 - DNA, 18 bp, synthetic construct
  source 1..18: mol_type = other DNA
aagaatgggaagggtgcc

SEQ ID NO: 93 - DNA, 18 bp, synthetic construct
  source 1..18: mol_type = other DNA
taagaatgggaagggtgc

SEQ ID NO: 94 - DNA, 18 bp, synthetic construct
  source 1..18: mol_type = other DNA
gtaagaatgggaagggtg

SEQ ID NO: 95 - DNA, 18 bp, synthetic construct
  source 1..18: mol_type = other DNA
agtaagaatgggaagggt

SEQ ID NO: 96 - DNA, 18 bp, synthetic construct
  source 1..18: mol_type = other DNA
gagtaagaatgggaaggg

SEQ ID NO: 97 - DNA, 18 bp, synthetic construct
  source 1..18: mol_type = other DNA
cagactttctcggtcata

SEQ ID NO: 98 - DNA, 18 bp, synthetic construct
  source 1..18: mol_type = other DNA
gcagactttctcggtcat

SEQ ID NO: 99 - DNA, 18 bp, synthetic construct
  source 1..18: mol_type = other DNA
tgcagactttctcggtca

SEQ ID NO: 100 - DNA, 18 bp, synthetic construct
  source 1..18: mol_type = other DNA
atgcagactttctcggtc

SEQ ID NO: 101 - DNA, 18 bp, synthetic construct
  source 1..18: mol_type = other DNA
aatgcagactttctcggt

SEQ ID NO: 102 - DNA, 18 bp, synthetic construct
  source 1..18: mol_type = other DNA
taatgcagactttctcgg

SEQ ID NO: 103 - DNA, 18 bp, synthetic construct
  source 1..18: mol_type = other DNA
gacctggacttttttccc

SEQ ID NO: 104 - DNA, 18 bp, synthetic construct
  source 1..18: mol_type = other DNA
tgacctggacttttttcc

SEQ ID NO: 105 - DNA, 18 bp, synthetic construct
  source 1..18: mol_type = other DNA
ctgacctggacttttttc

SEQ ID NO: 106 - DNA, 18 bp, synthetic construct
  source 1..18: mol_type = other DNA
gctgacctggactttttt

SEQ ID NO: 107 - DNA, 18 bp, synthetic construct
  source 1..18: mol_type = other DNA
ctgcaatgatgtggcaaa

SEQ ID NO: 108 - DNA, 18 bp, synthetic construct
  source 1..18: mol_type = other DNA
tctgcaatgatgtggcaa

SEQ ID NO: 109 - DNA, 18 bp, synthetic construct
  source 1..18: mol_type = other DNA
ttctgcaatgatgtggca

SEQ ID NO: 110 - DNA, 18 bp, synthetic construct
  source 1..18: mol_type = other DNA
cttctgcaatgatgtggc

SEQ ID NO: 111 - DNA, 18 bp, synthetic construct
  source 1..18: mol_type = other DNA
gctgcccacttgcatact

SEQ ID NO: 112 - DNA, 18 bp, synthetic construct
  source 1..18: mol_type = other DNA
gttggcagaacatagaac

SEQ ID NO: 113 - DNA, 18 bp, synthetic construct
  source 1..18: mol_type = other DNA
cgttggcagaacatagaa

SEQ ID NO: 114 - DNA, 18 bp, synthetic construct
  source 1..18: mol_type = other DNA
gcgttggcagaacataga

SEQ ID NO: 115 - DNA, 18 bp, synthetic construct
  source 1..18: mol_type = other DNA
atggggctacaggggata

SEQ ID NO: 116 - DNA, 18 bp, synthetic construct
  source 1..18: mol_type = other DNA
tatggggctacaggggat

SEQ ID NO: 117 - DNA, 18 bp, synthetic construct
  source 1..18: mol_type = other DNA
ttatggggctacagggga

SEQ ID NO: 118 - DNA, 18 bp, synthetic construct
  source 1..18: mol_type = other DNA
agttatggggctacaggg

SEQ ID NO: 119 - DNA, 18 bp, synthetic construct
  source 1..18: mol_type = other DNA
aagttatggggctacagg

SEQ ID NO: 120 - DNA, 18 bp, synthetic construct
  source 1..18: mol_type = other DNA
caagttatggggctacag

SEQ ID NO: 121 - DNA, 18 bp, synthetic construct
  source 1..18: mol_type = other DNA
ccaagttatggggctaca

SEQ ID NO: 122 - DNA, 18 bp, synthetic construct
  source 1..18: mol_type = other DNA
tccaagttatggggctac

SEQ ID NO: 123 - DNA, 18 bp, synthetic construct
  source 1..18: mol_type = other DNA
atccaagttatggggcta

SEQ ID NO: 124 - DNA, 18 bp, synthetic construct
  source 1..18: mol_type = other DNA
tatccaagttatggggct

SEQ ID NO: 125 - DNA, 18 bp, synthetic construct
  source 1..18: mol_type = other DNA
ctatccaagttatggggc

SEQ ID NO: 126 - DNA, 18 bp, synthetic construct
  source 1..18: mol_type = other DNA
attggaggaaatagggtg

SEQ ID NO: 127 - DNA, 18 bp, synthetic construct
  source 1..18: mol_type = other DNA
gtcttgtaggtagcagcc

SEQ ID NO: 128 - DNA, 18 bp, synthetic construct
  source 1..18: mol_type = other DNA
ggtcttgtaggtagcagc

SEQ ID NO: 129 - DNA, 18 bp, synthetic construct
  source 1..18: mol_type = other DNA
tggtcttgtaggtagcag

SEQ ID NO: 130 - DNA, 18 bp, synthetic construct
  source 1..18: mol_type = other DNA
ctggtcttgtaggtagca

SEQ ID NO: 131 - DNA, 18 bp, synthetic construct
  source 1..18: mol_type = other DNA
tctggtcttgtaggtagc

SEQ ID NO: 132 - DNA, 18 bp, synthetic construct
  source 1..18: mol_type = other DNA
gtctggtcttgtaggtag

SEQ ID NO: 133 - DNA, 18 bp, synthetic construct
  source 1..18: mol_type = other DNA
agtctggtcttgtaggta

SEQ ID NO: 134 - DNA, 18 bp, synthetic construct
  source 1..18: mol_type = other DNA
gagtctggtcttgtaggt

SEQ ID NO: 135 - DNA, 18 bp, synthetic construct
  source 1..18: mol_type = other DNA
ggagtctggtcttgtagg

SEQ ID NO: 136 - DNA, 18 bp, synthetic construct
  source 1..18: mol_type = other DNA
aggagtctggtcttgtag

(19) United States
(12) Patent Application Publication
(10) Pub. No.: US 2025/0270560 A1
(43) Pub. Date: Aug. 28, 2025

(54) ANTI-CANCER AGENTS

(71) Applicant: GMP BIOTECHNOLOGY LIMITED, Hong Kong (CN)

(72) Inventor: Vuong TRIEU, Agoura Hills, CA (US)

(21) Appl. No.: 19/201,626

(22) Filed: May 7, 2025

Related U.S. Application Data
(63) Continuation of application No. PCT/US2023/079075, filed on Nov. 8, 2023.
(60) Provisional application No. 63/471,168, filed on Jun. 5, 2023.
(60) Provisional application No. 63/451,220, filed on Mar. 9, 2023.
(60) Provisional application No. 63/384,088, filed on Nov. 16, 2022.
(60) Provisional application No. 63/383,453, filed on Nov. 11, 2022.
(60) Provisional application No. 63/383,032, filed on Nov. 9, 2022.

(51) Int. Cl. C12N 15/113; A61K 31/506; A61K 31/517; A61K 31/5377; A61K 38/20; A61K 45/06; A61P 35/00; C07K 16/28

(52) U.S. Cl. CPC C12N 15/1136; A61K 31/506; A61K 31/517; A61K 31/5377; A61K 38/2013; A61K 45/06; A61P 35/00; C07K 16/2818; C12N 2310/11; C12N 2310/14; C12N 2310/312; C12N 2310/315; C12N 2310/321; C12N 2310/322; C12N 2310/3231; C12N 2310/3233; C12N 2310/3341; C12N 2310/341; C12N 2310/3515

(57) ABSTRACT

This invention relates to agents, uses and methods for cancer designed to promote anti-tumor effects over a range of different cancers. Exemplary synergistic therapies include compositions of combinations of active agents including antisense agents for inhibiting or suppressing expression of TGF-β2, checkpoint inhibitor agents, and interleukin immunotherapeutic agents. One or more biomarkers can be used to select subjects who benefit from the agents, uses and methods, including IRF5 and ITGAM. The agents can be used with chemotherapy and other standard-of-care therapies.

### SEQUENCE LISTING

This application includes a sequence listing submitted electronically as an ST.26 file created on May 6, 2025, named 018988-005US1_SL.xml, which is 120,186 bytes in size.

### TECHNICAL FIELD

This invention describes agents, uses and methods for treating or ameliorating the symptoms of cancer in a human or animal subject. The agents are designed to promote anti-tumor effects over a range of different cancers. Exemplary synergistic therapies include various combinations of active agents including agents for inhibiting or suppressing expression of TGF-β2, checkpoint inhibitor agents, and interleukin immunotherapeutic agents. One or more biomarkers can be used to select subjects who benefit from the agents, uses, or methods including IRF5 and ITGAM. The therapies can be used in combination with chemotherapy, radiation therapy and other standard-of-care therapies.

### BACKGROUND

Cancer is a complex pathology involving multiple variant cellular pathways. Because of this complexity, it has been difficult to find effective therapeutic strategies that can have anti-tumor effects in various cancers.

Drawbacks of conventional therapies include lack of efficacy over a range of cancers.

Further drawbacks of conventional therapies include significant unwanted side effects such as killing healthy cells in addition to killing cancer cells.

Additional drawbacks of anti-cancer agents include high toxicity at required levels of therapeutic administration.

What is needed are methods, agents and uses for cancer diseases to increase efficacy, and reduce toxicity and unwanted side effects.

For example, what is needed are compositions, uses or methods with different agents in combination having significant anti-tumor effects and cancer immunotherapeutic effects, and which can reduce side effects and adverse health effects. There is a need for improved guidance of such compositions using appropriate biomarkers to select synergistic effects of the compositions.

There is an urgent need for new methods, agents and uses to combine strategies for cancer immunotherapy with strategies for direct anti-tumor attack for treating various cancers.

For example, what is needed are therapeutic compositions which combine cancer T-cell and immunotherapies with potent anti-cancer agents.

### BRIEF SUMMARY

This invention provides methods for treating or ameliorating the symptoms of cancer in a human or animal subject with pharmaceutical compositions designed to promote anti-tumor effects over a range of different cancers. Synergistic pharmaceutical therapies of this invention include use of potent, direct anti-tumor agents along with cancer immunotherapeutic agents. Cancer immunotherapeutics may include checkpoint inhibitor agents and protein immunotherapeutic agents. Methods, agents and uses of this invention may combine strategies for cancer immunotherapy with strategies for direct anti-tumor attack for treating various cancers.

In some embodiments, methods and therapeutic strategies of this invention can increase efficacy, as well as reduce toxic side effects and adverse health effects in cancer treatment.

In further embodiments, methods and therapeutic strategies of this invention can improve guidance of the therapy using appropriate biomarkers to select synergistic effects of the compositions.

Exemplary synergistic pharmaceutical therapies include compositions of various combinations of active agents including agents for inhibiting or suppressing expression of TGF-β2, checkpoint inhibitor agents, and interleukin immunotherapeutic agents. One or more biomarkers can be used to select subjects who benefit from the method, agent or use, including IRF5 and ITGAM. The compositions can be used in combination with chemotherapy and other standard-of-care therapies.

Embodiments of this invention include the following:

An antisense agent for inhibiting or suppressing expression of TGF-β2 in combination with a checkpoint inhibitor agent for use in treating or ameliorating the symptoms of cancer in a human subject or animal.

Use of an antisense agent for inhibiting or suppressing expression of TGF-β2 in the preparation of a medicament for treating or ameliorating the symptoms of a cancer in a human subject or animal in combination with a checkpoint inhibitor agent.

A method for treating or ameliorating the symptoms of cancer in a human or animal subject in need, the method comprising:
administering a therapeutically effective amount of an antisense agent for inhibiting or suppressing expression of TGF-β2 to the subject;
administering a therapeutically effective amount of a checkpoint inhibitor agent to the subject.

The agent above, in combination with an interleukin immunotherapeutic agent.

The use above, in combination with an interleukin immunotherapeutic agent.

The method above, comprising administering a therapeutically effective amount of an interleukin immunotherapeutic agent to the subject.

The agent, use or method above, wherein the agent for inhibiting or suppressing expression of TGF-β2, the checkpoint inhibitor, and the interleukin immunotherapeutic agent are administered concurrently, simultaneously, sequentially, or separately in time.

The agent, use or method above, wherein the agent for inhibiting or suppressing expression of TGF-β2, the checkpoint inhibitor, and the interleukin immunotherapeutic agent are administered separately or in combined formulation by injection or infusion.

The agent, use or method above, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

The agent, use or method above, wherein the agent for inhibiting or suppressing expression of TGF-β2 is a TGF-β2-specific antisense oligonucleotide complementary to a TGF-β2 transcript and 15-30 nucleotides in length.

The agent, use or method above, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

The agent, use or method above, wherein the agent for inhibiting or suppressing expression of TGF-β2 is one or more TGF-β2-specific antisense oligonucleotides as shown in Table 1, complementary to a TGF-β2 transcript, and chemically-modified variants thereof, LNA variants thereof, gapmer variants thereof, and any combination or pooling thereof.

The agent, use or method above, wherein the TGF-β2-specific antisense oligonucleotides have no more than one or two mismatches as compared to a target human TGF-β2.

The agent, use or method above, wherein the TGF-β2-specific antisense oligonucleotides reduce a TGF-β2 transcript level by at least 60%, or at least 70%, or at least 80%, or at least 90%.

The agent, use or method above, wherein the TGF-β2-specific antisense oligonucleotides reduce any TGF-β1 transcript level and any TGF-β3 transcript level by less than 10%, or less than 5%, or less than 1%.

The agent, use or method above, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the antisense agent is conjugated to a polyethylene glycol, a lipid, or a triantenarry N-acteyl-galactosamine.

The agent, use or method above, wherein each agent comprises a carrier of sterile water for injection, saline, isotonic saline, or a combination thereof, which may be the same or different for each agent.

The agent, use or method above, wherein the agents are substantially free of excipients.

The agent, use or method above, wherein the agents are stable in a carrier substantially free of excipients for at least 14 days at 37° C.

The agent, use or method above, wherein the checkpoint inhibitor agent is an inhibitor of PD-1.

The agent, use or method above, wherein the checkpoint inhibitor agent is pembrolizumab, nivolumab, cemiplimab, spartalizumab, atezolizumab, avelumab, or durvalumab.

The agent, use or method above, wherein the interleukin immunotherapeutic agent is a natural IL-2, a high dose IL-2, a recombinant IL-2, or aldesleukin.

The agent, use or method above, comprising selecting subjects who benefit from the agent, use or method based on levels of one or more biomarkers TGF-B2, IL-2, CD19, IRF5, ITGAM, and a combination thereof.

The agent, use or method above, wherein the one or more biomarkers is IRF5 and the subject is selected when expression of IRF5 is at a level above the median.

The agent, use or method above, wherein the one or more biomarkers is ITGAM and the subject is selected when expression of ITGAM is at a level above the median.

The agent, use or method above, wherein the subject after the administration or use has a decreased level of TGF-β2 as compared to before the administration or use.

The agent, use or method above, wherein the subject after the administration or use has an increased level of IRF5 as compared to before the administration or use.

The agent, use or method above, wherein the subject after the administration or use has a decreased level of ITGAM as compared to before the administration or use.

The agent, use or method above, comprising administering a therapeutically effective amount of an expression product of IRF5 or ITGAM to the subject.

The agent, use or method above, wherein the expression product is an mRNA, polypeptide, protein, or fragment thereof, or combination thereof.

The agent, use or method above, wherein the administration or use decreases mortality rate at month 6, 12, 18, 24, 30, or 36.

The agent, use or method above, wherein the administration or use increases overall survival rate at month 6, 12, 18, 24, 30, or 36.

The agent, use or method above, in combination with any one or more medicaments comprising a targeted cancer drug, a cancer growth blocker, an EGFR inhibitor, and combinations thereof.

The agent, use or method above, in combination with any one or more medicaments selected from bevacizumab, everolimus, belzutifan, dabrafenib, trametinib, and combinations thereof.

The agent, use or method above, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, in combination with any one or more medicaments which are EGFR inhibitors selected from erlotinib, gefitinib, afatinib, osimertinib, dacomitininb, and combinations thereof.

The agent, use or method above, in combination with a chemotherapy medicament.

The agent, use or method above, in combination with radiation therapy or electric field therapy.

An antisense agent for inhibiting or suppressing expression of TGF-β2 in combination with an interleukin immunotherapeutic agent for use in treating or ameliorating the symptoms of cancer in a human subject or animal.

Use of an antisense agent for inhibiting or suppressing expression of TGF-β2 in the preparation of a medicament for treating or ameliorating the symptoms of a cancer in a human subject or animal in combination with an interleukin immunotherapeutic agent.

A method for treating or ameliorating the symptoms of cancer in a human or animal subject in need, the method comprising:
administering a therapeutically effective amount of an antisense agent for inhibiting or suppressing expression of TGF-β2 to the subject;
administering a therapeutically effective amount of an interleukin immunotherapeutic agent to the subject.

The agent, use or method above, wherein the agent for inhibiting or suppressing expression of TGF-β2, and the interleukin immunotherapeutic agent are administered concurrently, simultaneously, sequentially, or separately in time.

The agent, use or method above, wherein the agent for inhibiting or suppressing expression of TGF-β2, and the interleukin immunotherapeutic agent are administered separately or in combined formulation by injection or infusion.

The agent, use or method above, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

The agent, use or method above, wherein the agent for inhibiting or suppressing expression of TGF-β2 is a TGF-β2-specific antisense oligonucleotide complementary to a TGF-β2 transcript and 15-30 nucleotides in length.

The agent, use or method above, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

The agent, use or method above, wherein the agent for inhibiting or suppressing expression of TGF-β2 is one or more TGF-β2-specific antisense oligonucleotides as shown in Table 1, complementary to a TGF-β2 transcript.

The agent, use or method above, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the antisense agent is conjugated to a polyethylene glycol, a lipid, or a triantenarry N-acteyl-galactosamine.

The agent, use or method above, wherein each agent comprises a carrier of sterile water for injection, saline, isotonic saline, or a combination thereof, which may be the same or different for each agent.

The agent, use or method above, wherein the agents are substantially free of excipients.

The agent, use or method above, wherein the agents are stable in a carrier substantially free of excipients for at least 14 days at 37° C.

The agent, use or method above, wherein the interleukin immunotherapeutic agent is a natural IL-2, a high dose IL-2, a recombinant IL-2, or aldesleukin.

The agent, use or method above, wherein the administration or use decreases mortality rate at month 6, 12, 18, 24, 30, or 36.

The agent, use or method above, wherein the administration or use increases survival rate at month 6, 12, 18, 24, 30, or 36.

The agent, use or method above, in combination with any one or more medicaments comprising a targeted cancer drug, a cancer growth blocker, an EGFR inhibitor, and combinations thereof.

The agent, use or method above, in combination with any one or more medicaments selected from bevacizumab, everolimus, belzutifan, dabrafenib, trametinib, and combinations thereof.

The agent, use or method above, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, in combination with any one or more medicaments which are EGFR inhibitors selected from erlotinib, gefitinib, afatinib, osimertinib, dacomitininb, and combinations thereof.

The agent, use or method above, in combination with a chemotherapy medicament.

The agent, use or method above, in combination with radiation therapy or electric field therapy.

The agent, use or method above, comprising selecting subjects who benefit from the agent, use or method based on levels of one or more biomarkers TGF-β2, IRF5, ITGAM, and a combination thereof.

The agent, use or method above, comprising selecting subjects who benefit from the agent, use or method based on levels of one or more biomarkers TGF-β2, IRF5, ITGAM, neoantigen, mutational load, macrophage and a combination thereof.

The agent, use or method above, wherein the one or more biomarkers is IRF5 and the subject is selected when expression of IRF5 is at a level below the median.

The agent, use or method above, wherein the one or more biomarkers is tumor associated macrophage and the subject is selected when tumor associated macrophage is below average.

The agent, use or method above, wherein the one or more biomarkers is tumor neoantigen mutation load and the subject is selected when neoantigen tumor load is below average.

The agent, use or method above, comprising administering a therapeutically effective amount of an agent for inhibiting or suppressing expression of ITGAM or IRF5 to the subject.

The agent, use or method above, wherein the agent for inhibiting or suppressing expression of ITGAM or IRF5 is an antisense oligonucleotide targeted to ITGAM or IRF5, respectively.

A kit for treating or ameliorating the symptoms of cancer, the kit comprising:
a therapeutically effective amount of an antisense agent for inhibiting or suppressing expression of TGF-β2; and
a therapeutically effective amount of a checkpoint inhibitor agent.

The kit above, comprising a therapeutically effective amount of an interleukin immunotherapeutic agent.

The kit above, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, a thymus cancer, or a multiple myeloma.

The kit above, wherein the agent for inhibiting or suppressing expression of TGF-β2 is a TGF-β2-specific antisense oligonucleotide complementary to a TGF-β2 transcript and 15-30 nucleotides in length.

The kit above, wherein the agent for inhibiting or suppressing expression of TGF-2 is a TGF-β2-specific antisense oligonucleotides complementary to a TGF-β2 pre-RNA, pre-mRNA or mRNA and 18-21 nucleotides in length.

The kit above, wherein the agent for inhibiting or suppressing expression of TGF-β2 is one or more TGF-β2-specific antisense oligonucleotides as shown in Table 1, complementary to a TGF-β2 transcript.

The kit above, wherein the TGF-β2-specific antisense oligonucleotides have no more than one or two mismatches as compared to a target human TGF-2.

The kit above, wherein the TGF-β2-specific antisense oligonucleotides reduce a TGF-β2 transcript level by at least 60%, or at least 70%, or at least 80%, or at least 90%.

The kit above, wherein the TGF-β2-specific antisense oligonucleotides reduce any TGF-β1 transcript level and any TGF-β3 transcript level by less than 10%, or less than 5%, or less than 1%.

The kit above, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The kit above, wherein the antisense agent is conjugated to a polyethylene glycol, a lipid, or a triantenarry N-acteyl-galactosamine.

The kit above, wherein each agent comprises a carrier of sterile water for injection, saline, isotonic saline, or a combination thereof, which may be the same or different for each agent.

The kit above, wherein the agents are substantially free of excipients.

The kit above, wherein the agents are stable in a carrier substantially free of excipients for at least 14 days at 37° C.

### BRIEF DESCRIPTION OF THE DRAWINGS

FIG. 1 shows Kaplan-Meier overall survival charts for a study of such clinical effects. FIG. 1 shows that for use of a PD-1 inhibitor, improved survival was indicated for high IL2 (left panel), which can be provided with an IL-2 immunotherapy agent, and low TGF-β2 (right panel), which can be provided with an antisense TGF-β2 inhibitor. These clinical data showed basis for a therapeutic combination of an antisense TGF-β2 inhibitor, a PD-1 checkpoint inhibitor, and an IL-2 immunotherapy agent for improved overall survival of melanoma patients.

FIG. 2 shows that for use of a PD-1 inhibitor, improved survival is highly indicated for a higher ratio of IL-2/TGF-β2 (left panel), which can be provided with an IL-2 immunotherapy agent and an antisense TGF-β2 inhibitor. These clinical data showed basis for a therapeutic combination of an antisense TGF-β2 inhibitor, a PD-1 checkpoint inhibitor, and an IL-2 immunotherapy agent for improved overall survival of melanoma patients.

FIG. 3 shows data for biomarker use of ITGAM. FIG. 3 (upper left, right panels) show Kaplan-Meier overall survival charts for this study of melanoma patients. FIG. 3 (lower left, right panels) show Kaplan-Meier overall survival charts for this study of pancreatic cancer patients (PDAC) through the lens of TGF-β2 expression.

FIG. 4 shows data for biomarker use of ITGAM. FIG. 4 (left, right panels) show Kaplan-Meier overall survival charts for this study of pancreatic cancer patients (PDAC) through the lens of IL-2 expression.

FIG. 5 shows data for biomarker use of CD8A. FIG. 5 (upper left, right panels) show Kaplan-Meier overall survival charts for this study of melanoma patients. FIG. 5 (lower left, right panels) show Kaplan-Meier overall survival charts for this study of pancreatic cancer patients (PDAC) through the lens of TGF-β2 expression.

FIG. 6 shows data for biomarker use of CD8A. FIG. 6 (left, right panels) show Kaplan-Meier overall survival charts for this study of pancreatic cancer patients (PDAC) through the lens of IL-2 expression.

FIG. 7 shows data for biomarker use of CD4. FIG. 3 (upper left, right panels) show Kaplan-Meier overall survival charts for this study of melanoma patients. FIG. 7 (lower left, right panels) show Kaplan-Meier overall survival charts for this study of pancreatic cancer patients (PDAC) through the lens of TGF-β2 expression.

FIG. 8 shows data for biomarker use of CD4. FIG. 8 (left, right panels) show Kaplan-Meier overall survival charts for this study of pancreatic cancer patients (PDAC) through the lens of IL-2 expression.

FIG. 9 shows data for biomarker use of ITGAX. FIG. 9 (upper left, right panels) show Kaplan-Meier overall survival charts for this study of melanoma patients. FIG. 9 (lower left, right panels) show Kaplan-Meier overall survival charts for this study of pancreatic cancer patients (PDAC) through the lens of TGF-β2 expression.

FIG. 10 shows data for biomarker use of ITGAX. FIG. 10 (left, right panels) show Kaplan-Meier overall survival charts for this study of pancreatic cancer patients (PDAC) through the lens of IL-2 expression.

FIG. 11 shows data for biomarker use of CD19. FIG. 11 (upper left, right panels) show Kaplan-Meier overall survival charts for this study of melanoma patients. FIG. 11 (lower left, right panels) show Kaplan-Meier overall survival charts for this study of pancreatic cancer patients (PDAC) through the lens of TGF-β2 expression.

FIG. 12 shows data for biomarker use of CD19. FIG. 12 (left, right panels) show Kaplan-Meier overall survival charts for this study of pancreatic cancer patients (PDAC) through the lens of IL-2 expression.

FIG. 13 shows data for biomarker use of IRF5. FIG. 13 (upper left, right panels) show Kaplan-Meier overall survival charts for this study of melanoma patients. FIG. 13 (lower left, right panels) show Kaplan-Meier overall survival charts for this study of pancreatic cancer patients (PDAC) through the lens of TGF-β2 expression.

FIG. 14 shows data for biomarker use of IRF5. FIG. 14 (left, right panels) show Kaplan-Meier overall survival charts for this study of pancreatic cancer patients (PDAC) through the lens of IL-2 expression.

FIG. 15 shows data for biomarker use of NOS2. FIG. 15 (upper left, right panels) show Kaplan-Meier overall survival charts for this study of melanoma patients. FIG. 15 (lower left, right panels) show Kaplan-Meier overall survival charts for this study of pancreatic cancer patients (PDAC) through the lens of TGF-β2 expression.

FIG. 16 shows data for biomarker use of NOS2. FIG. 16 (left, right panels) show Kaplan-Meier overall survival charts for this study of pancreatic cancer patients (PDAC) through the lens of IL-2 expression.

FIG. 17 shows data for biomarker use of CD163. FIG. 17 (upper left, right panels) show Kaplan-Meier overall survival charts for this study of melanoma patients. FIG. 17 (lower left, right panels) show Kaplan-Meier overall survival charts for this study of pancreatic cancer patients (PDAC) through the lens of TGF-β2 expression.

FIG. 18 shows data for biomarker use of CD163. FIG. 18 (left, right panels) show Kaplan-Meier overall survival charts for this study of pancreatic cancer patients (PDAC) through the lens of IL-2 expression.

FIG. 19 shows the results of this clinical study of overall survival of melanoma patients. FIG. 19 shows Kaplan-Meier overall survival charts for a study of such clinical effects. FIG. 19 shows that for use of a PD-1 checkpoint inhibitor, with additional stratification based on various immune cell indicators, namely basophils, B cells, eosinophils, and M0 macrophages, and Th1 helper cells, a high level of IL-2 significantly increased survival in all cases. M0 macrophages were a highly significant factor.

FIG. 20 shows the results of this clinical study of overall survival of melanoma patients. FIG. 20 shows Kaplan-Meier overall survival charts for a study of such clinical effects. FIG. 20 shows that for use of a PD-1 checkpoint inhibitor, with additional stratification based on various immune cell indicators, namely basophils, B cells, eosinophils, and M0 macrophages, and Th1 helper cells, a high level of IL-2 significantly increased survival in all cases. FIG. 20 (upper right panel and lower panel) shows comparative baseline results.

FIG. 21 shows the results of this clinical study of overall survival of pancreatic cancer patients. FIG. 21 shows Kaplan-Meier overall survival charts for a study of such clinical effects. FIG. 21 (upper left panel) shows that for patients with low M2 tumor-associated-macrophage, a high tumoral mRNA level of TGF-β2 significantly decreased survival. Logrank P value in FIG. 21 (upper left panel) indicates high significance for improved survival using a therapeutic antisense TGF-β2 inhibitor based on this clinical study and conditions. Survival of patients in the high range of tumoral TGF-β2 expression was only 15 months, as compared to 73 months for patients in the low range of tumoral TGF-β2 expression.

FIG. 22 shows Kaplan-Meier overall survival charts for a study of clinical effects of combination cancer therapy.

FIG. 23 shows Kaplan-Meier overall survival charts for a study of clinical effects of combination cancer therapy.

FIG. 24 shows Kaplan-Meier overall survival charts for a study of clinical effects of combination cancer therapy.

FIG. 25 shows Kaplan-Meier overall survival charts for a study of clinical effects of combination cancer therapy.

FIG. 26 shows Kaplan-Meier overall survival charts for a study of clinical effects of combination cancer therapy.

FIG. 27 shows Kaplan-Meier overall survival charts for a study of clinical effects of combination cancer therapy.

FIG. 28 shows Kaplan-Meier overall survival charts for a study of clinical effects of combination cancer therapy.

FIG. 29 shows Kaplan-Meier overall survival charts for a study of clinical effects of combination cancer therapy.

FIG. 30 shows Kaplan-Meier overall survival charts for a study of clinical effects of combination cancer therapy.

FIG. 31 shows Kaplan-Meier overall survival charts for a study of clinical effects of combination cancer therapy.

FIG. 32 shows Kaplan-Meier overall survival charts for a study of clinical effects of combination cancer therapy.

FIG. 33 shows Kaplan-Meier overall survival charts for a study of clinical effects of combination cancer therapy.

FIG. 34 shows Kaplan-Meier overall survival charts for a study of clinical effects of combination cancer therapy.

FIG. 35 shows Kaplan-Meier overall survival charts for a study of clinical effects of combination cancer therapy.

FIG. 36 shows Kaplan-Meier overall survival charts for a study of clinical effects of combination cancer therapy.

FIG. 37 shows Kaplan-Meier overall survival charts for a study of clinical effects of combination cancer therapy.

FIG. 38 shows Kaplan-Meier overall survival charts for a study of clinical effects of combination cancer therapy.

FIG. 39 shows Kaplan-Meier overall survival charts for a study of clinical effects of combination cancer therapy.

FIG. 40 shows Kaplan-Meier overall survival charts for a study of clinical effects of combination cancer therapy.

FIG. 41 shows Kaplan-Meier overall survival charts for a study of clinical effects of combination cancer therapy.

FIG. 42 shows Kaplan-Meier overall survival charts for a study of clinical effects of combination cancer therapy.

FIG. 43 shows Kaplan-Meier overall survival charts for a study of clinical effects of combination cancer therapy.

FIG. 44 shows Kaplan-Meier overall survival charts for a study of clinical effects of combination cancer therapy.

FIG. 45 shows Kaplan-Meier overall survival charts for a study of clinical effects of combination cancer therapy.

### DETAILED DESCRIPTION OF THE DISCLOSURE

This invention relates to methods, compositions, agents and therapeutic uses thereof for treating or ameliorating the symptoms of cancer in a human or animal subject with pharmaceutical compositions designed to promote anti-tumor effects over a range of different cancers.

Exemplary synergistic pharmaceutical therapies include compositions of various combinations of active agents including agents for inhibiting or suppressing expression of TGF-β2, checkpoint inhibitor agents, and interleukin immunotherapeutic agents.

Embodiments of this invention include methods, agents and therapeutic uses thereof for treating or ameliorating symptoms of oncological disease in which agents may be administered concurrently, simultaneously, sequentially, or separately in time.

In certain embodiments, a highly stable formulation of one or more anti-TGF-β2 agents may be used for oncological disease in combination with one or more immunotherapeutic agents, in which the anti-TGF-β2 agents and the immunotherapeutic agents are used concurrently, simultaneously, sequentially, or separately in time.

In some embodiments, one or more biomarkers can be used to select subjects who benefit from the method, agent or use, including IRF5 and ITGAM. The compositions can be used in combination with chemotherapy and other standard-of-care therapies.

In some embodiments, this invention contemplates a combination of immunotherapeutic agents including checkpoint inhibitors with TGF-β2 inhibitors as guided by biomarkers.

Embodiments of this invention encompass methods, agents and uses for immunotherapeutic agents including checkpoint inhibitors in combination with TGF-β2 inhibitors and guided by biomarkers. These embodiments recognize that overexpression of TGF-β2 is a useful indicator to avoid a cascade of downstream effects and poor outcomes in oncological disease.

This invention can utilize detection of TGF-β2 biomarkers as a guide to select subjects for therapy with immunotherapeutic agents including checkpoint inhibitors in combination with TGF-β2 inhibitors. Because antisense oligonucleotides of this invention, such as OT-101, target and inhibit TGF-β2, the selection of subjects for therapy using TGF-β2 biomarkers advantageously provides improved outcomes.

More particularly, this invention may utilize TGF-β2 as biomarker, which is surprisingly superior to TGF-beta-1 or TGF-beta-3 for outcomes in oncological disease. TGF-β2 as a biomarker is predictive for improved outcomes, whereas TGF-beta-1 or TGF-beta-3 are not and may indicate poorer outcomes.

In some embodiments, the combination of a TGF-β2 antisense inhibitor with a PD-1 checkpoint inhibitor can be surprisingly effective. In certain embodiments, the combination of a TGF-β2 antisense inhibitor with a PD-1 checkpoint inhibitor is surprisingly efficacious because the combination of a TGF-β2 antisense inhibitor with a PD-L1 checkpoint inhibitor is not.

Embodiments of this invention utilize these facts to provide methods, agents or uses for oncological disease by selecting a subject using a TGF-β2 biomarker, wherein the subject is selected when expression of TGF-β2 is elevated.

In some embodiments, methods for treating or ameliorating the symptoms of cancer in a human subject or animal subject in need may comprise administering a therapeutically sufficient amount of a pharmaceutical composition comprising an agent for inhibiting or suppressing expression of TGF-β2 to the subject, and administering a therapeutically sufficient amount of a pharmaceutical composition comprising a checkpoint inhibitor to the subject, and administering a therapeutically sufficient amount of a pharmaceutical composition comprising an interleukin immunotherapeutic agent to the subject in combination, where the subject is selected using a TGF-β2 biomarker, and where the subject is selected when expression of TGF-β2 is elevated.

In further embodiments, an agent for inhibiting or suppressing expression of TGF-β2 in combination with a checkpoint inhibitor and an interleukin immunotherapeutic agent for use in treating or ameliorating the symptoms of cancer in a human subject or animal may be used for oncological disease by selecting the subject using a TGF-β2 biomarker, where the subject is selected when expression of TGF-2 is elevated.

Therapies of this invention may be applied for a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

As used herein, the term agent can refer to one or more active compounds, a combination of active compounds, or a composition containing one or more active compounds and a carrier, and/or a solvent, and/or any number of excipients. In some embodiments, the composition may be a pharmaceutical composition. In certain embodiments, the composition may be a pharmaceutical composition containing a therapeutically effective amount of one or more active compounds. Some examples of excipients are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N. Y., 1980. Methods for determining a therapeutically effective amount of a compound are known in the art.

### Anti-Cancer Agents and Methods

Many cancers such as pancreatic cancer (PDAC), melanoma, and others present patients with high levels of expression of TGF-β2. Agents for inhibiting or suppressing expression of TGF-β2 can be effective in treating these cancer types. For example, in patients with pancreatic cancer (PDAC), overall survival time more than doubles from 15 months for high TGF-β2 patients to 37 months for low TGF-β2 patients.

This invention includes methods for treating or ameliorating the symptoms of cancer in a human or animal subject in need, by administering a therapeutically sufficient amount of a pharmaceutical composition comprising an agent for inhibiting or suppressing expression of TGF-β2 to the subject; administering a therapeutically sufficient amount of a pharmaceutical composition comprising a checkpoint inhibitor to the subject; and administering a therapeutically sufficient amount of a pharmaceutical composition comprising an interleukin immunotherapeutic agent to the subject.

In certain embodiments, this invention includes agents for inhibiting or suppressing expression of TGF-β2 in combination with a checkpoint inhibitor for use in treating or ameliorating the symptoms of cancer in a human subject or animal.

In further embodiments, this invention includes agents for inhibiting or suppressing expression of TGF-β2 in combination with an interleukin immunotherapeutic agent for use in treating or ameliorating the symptoms of cancer in a human subject or animal.

In additional embodiments, this invention includes agents for inhibiting or suppressing expression of TGF-β2 in combination with a checkpoint inhibitor and an interleukin immunotherapeutic agent for use in treating or ameliorating the symptoms of cancer in a human subject or animal.

This invention further contemplates uses of a composition comprising an agent for inhibiting or suppressing expression of TGF-β2 in the preparation of a medicament for treating or ameliorating the symptoms of a cancer in a human subject or animal in combination with a checkpoint inhibitor and/or an interleukin immunotherapeutic agent.

Therapies of this invention using one or more agents for inhibiting or suppressing expression of TGF-β2 may be applied for a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

Examples of agents for inhibiting or suppressing expression of TGF-β2 include antisense agents.

### Human TGF-β2-Specific Antisense Oligodeoxynucleotide Agents

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA

oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide, for example OT-101, which is AP 12009 Trabedersen SEQ ID NO:8, can be used to reduce the level of TGF-β2 protein in malignancies, and delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C177H208N60Na17094P17S17 and the molecular weight 6,143 g/mol. OT-101 was designed to be complementary to a specific sequence of human TGF-2 mRNA following expression of the gene.

Antisense oligodeoxynucleotides are short strings of DNA that are designed to downregulate gene expression by interfering with the translation of a specific encoded protein at the mRNA level. For example, SEQ ID NO:8 (OT-101) is a synthetic 18-mer phosphorothioate oligodeoxynucleotide (S-ODN) in which a non-bridging oxygen of each phosphate moiety is substituted by a sulfur atom. OT-101 is complementary to a specific sequence of human TGF-β2 mRNA from expression of the gene. OT-101 can be an RNA therapeutic designed to abrogate the immunosuppressive actions of TGF-β2 and reduce the level of TGF-β2 in malignancies, to treat or ameliorate the symptoms of cancer, or delay the progression of disease.

A target TGF-β2 mRNA can be NCBI Reference Sequence: NM_003238.3 of sequence length 5,882 bp. A target region for TGF-β2 mRNA can be the protein coding sequence from reference 1,369 to 2,613.

Examples of agents of this disclosure for inhibiting or suppressing expression of TGF-β2 include TGF-β2-specific antisense oligonucleotides given in SEQ ID NOs: 1-136 in Table 1.

TABLE 1
TGF-β2-specific antisense oligonucleotides

| SEQ ID | | |
|---|---|---|
| NO: | Ref. | ANTISENSE SEQUENCE |
| 1 | — | GTTCGTTTAG AGAACAGATC |
| 2 | — | TAAAGTTCGT TTAGAGAACA G |
| 3 | — | AGCCCTGTAT ACGAC |
| 4 | — | GTAGGTAAAA ACCTAATAT |
| 5 | — | CGTTTAGAGA ACAGATCTAC |
| 6 | — | CATTGTAGAT GTCAAAAGCC |
| 7 | — | CTCCCTCATG GTGGCAGTTG A |
| 8 | — | CGGCATGTCT ATTTTGTA |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

The sequences of Table 1 can be chemically-modified to provide active variants thereof, LNA variants thereof, as well as gapmer variants thereof, as known in the art. The sequences of Table 1 can be used in any combination as active agents, such as pooling combinations.

It is understood that additional antisense oligonucleotides can be constructed based on the TGF-β2 gene sequence.

In some embodiments, a TGF-β2-specific antisense oligonucleotide of this invention may have no more than one or two mismatches as compared to a target human TGF-β2.

In certain embodiments, a TGF-β2-specific antisense oligonucleotide of this invention may reduce a TGF-β2 transcript level by at least 60%, or at least 70%, or at least 80%, or at least 90%.

In additional embodiments, a TGF-β2-specific antisense oligonucleotide of this invention may be selective for TGF-β2 and reduce any TGF-β1 transcript level and any TGF-β3 transcript level by less than 10%, or less than 5%, or less than 1%.

In further embodiments, a therapeutically effective amount of an antisense agent for inhibiting or suppressing expression of TGF-β2 can be from 0.1 to 3000 mg per day, or 1 to 1000 mg per day, or 2 to 500 mg per day, or 2 to 200 mg per day.

In certain embodiments, a formulation of an antisense agent for inhibiting or suppressing expression of TGF-β2 can have a concentration of from 0.05 to 50 μM, or 0.1 to 25 μM, or 0.1 to 10 μM, or 0.1 to 7.5 μM, or 0.1 to 5 μM.

In certain embodiments, a method for using an antisense agent for inhibiting or suppressing expression of TGF-β2 can use an effective dosage amount of from 1 to 1000 mg/m²/day, or from 1 to 500 mg/m²/day, or from 1 to 250 mg/m²/day, or from 1 to 100 mg/m²/day, or from 1 to 50 mg/m²/day. Mean human body surface area can be about 1.6 to 1.9 m².

In additional embodiments, a method for using an antisense agent for inhibiting or suppressing expression of TGF-β2 can use an effective dosage amount of from 0.05 to 40 mg/kg/day, or from 0.1 to 30 mg/kg/day, or from 0.2 to 20 mg/m²/day, or from 0.3 to 10 mg/m²/day, or from 0.5 to 5 mg/m²/day. Mean human body weight can be about 60 kg.

In certain embodiments, agents of this disclosure for inhibiting or suppressing expression of TGF-β2 may be prepared from a lyophilized powder of the agent.

In some examples and embodiments, an agent may be a TGF-β2-specific antisense oligonucleotide selected from SEQ ID NOs: 1-136, and administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7. In some embodiments, an agent may be a TGF-β2-specific antisense oligonucleotide selected from SEQ ID NOs: 9-136, as chemically-modified and administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7.

In certain embodiments, OT-101 may be supplied as a sterile lyophilizate for solution prior to administration in 20R glass vials with a quantity of 250 mg/vial. The lyophilizate may be reconstituted aseptically in sterile, preservative-free isotonic NaCl solution. OT-101 solution can be administered every 14 days using a portable pump system as a continuous i.v. infusion on days 4-7 according to a 4-days-on, 10-days-off schedule. A Schedule may be 7 d on/7d off and 4 d on/10 d off scheduling. A Dose may be 40, 80, 160, 140, 190, 250, 330 mg.

In certain embodiments, an agent may be a TGF-β2 gene sequence.-specific antisense oligonucleotide selected from SEQ ID NOs: 1-136, and administered or used by injection or infusion at a dose of 40, 80, 160, 140, 190, 250, 330 mg/m² on Days 1 to 7, or at a dose of 40, 80, 160, 140, 190, 250, 330 mg/m² on Days 1 to 4.

In some examples and embodiments, an agent may be a TGF-β2 gene sequence.-specific antisense oligonucleotide selected from SEQ ID NOs: 1-136, and administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

In some embodiments, an agent may be a TGF-β2 gene sequence-specific antisense oligonucleotide selected from SEQ ID NOs: 9-136, and administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

A therapeutically effective amount can also be determined with routine experimentation, for example, by monitoring a subject's response to administration of an agent and adjusting the dosage. See for example, Remington, The Science and Practice of Pharmacy (Gennaro ed. 20th edition) (2000).

Embodiments of this invention involving administration or use of a composition of an agent can ameliorate or suppress symptoms due to TGF-β2 induced proteins.

Embodiments of this invention further include pharmaceutical compositions for inhibiting or suppressing expression of TGF-β2, or for treating or ameliorating the symptoms of cancer in a human or animal. The pharmaceutical compositions may contain a TGF-β2 inhibitor, pharmaceutically acceptable salts forms, esters, polymorphs or stereoisomers thereof, and any combination thereof, as well as a carrier. The TGF-β2 inhibitor may be selected from TGF-β2-specific antisense oligonucleotides SEQ ID NOs: 1-136, or SEQ ID NOs: 9-136, and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

Agents of this disclosure may be diluted for formulation into admixtures for administration by infusion in components such as intravenous bags, syringes, and tubing, as are known in the art. Such formulations may contain multiple agents, as well as excipients.

Embodiments of this invention further contemplate therapeutic modalities in which a composition of this invention is administered or utilized in combination with a standard of care therapy for the disease. Examples of additional medicaments which may be administered or utilized in combination with a composition of this invention include anti-inflammatoires, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

Some TGF-β2-specific antisense oligonucleotide agents are given in U.S. Pat. Nos. 9,963,703, 9,758,786, and 8,476,246.

For example, the API trabedersen (OT-101) is a synthetic 18-mer S-ODN comprised of the bases adenine (A), thymine (T), guanine (G), and cytosine (C), with all 3′-5′ linkages modified to phosphorothioates. This sulfur modification makes the drug more resistant to degradation, resulting in an increased stability in vitro and in vivo. Its primary molecular structure, the nucleotide sequence, was designed to be complementary to a specific sequence of human transforming growth factor-beta 2 (TGF-β2) mRNA. This sequence and related sequences can be used for superior chemical and structural properties, biological activity, and specificity to achieve the best antisense effects in vitro and in vivo.

Chemical structure, exemplary of the phosphorothioate moieties (C-A-G), and the physical characteristics of trabedersen are shown in Table 2.

TABLE 2
Chemical and Physical Characteristics of Trabedersen (OT-101)

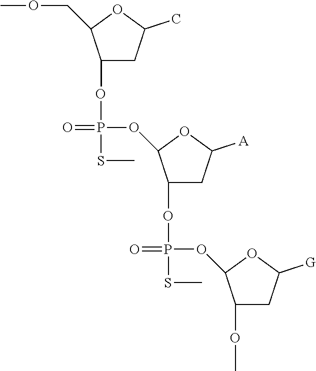

Phosphorothioate linkages
(C—A—G)

| | Molecular | Molecular | Number | | | | | |
|---|---|---|---|---|---|---|---|---|
| | Weight (sodium | Formula (sodium | of | Backbone | | Water | | pH of |
| Appearance: | salt, anhydrous): | salt, anhydrous): | moieties: | characteristics: | Polymorphism: | content: | Solubility: | solution: |
| White to | C₁₇₇N₂₀₈N₆₀O₉₄P₁₇S₁₇Na₁₇ | 6143 g/mol | 18 | Phosphorothioates | No crystalline | Up to | Very soluble in | 6-8 |
| off-white | | | | | morphology | 15% | water (>50 mg/mL) | |
| solid | | | | | in the solid | | Soluble in methanol | |
| | | | | | | | (>8.5 mg/mL) | |
| | | | | | | | Insoluble in iso- | |
| | | | | | | | propanol | |
| | | | | | | | (0.01 mg/mL) | |

The investigational medicinal product can be supplied as a sterile lyophilizate for solution for infusion in 50 mL glass vials (primary container) containing 7.37 mg trabedersen (intratumoral treatment) and in 20R glass vials (primary container) containing 250 mg trabedersen (intravenous treatment), respectively. The finished drug product may contain no excipients. Glass vials may be used for parenterals. Sterile rubber stoppers appropriate for lyophilization can seal the glass vial. The stopper may be sealed with a crimping capsule that includes a colored flip-off cap. For clinical use, each vial can be provided within a white-colored folding box to protect the vials from light exposure and damage during transport. Both the glass vials and the folding boxes may be labeled according to local requirements. The primary as well as secondary containers of the closure system can fulfill international quality standards for the packaging of sterile solid drug products for injections.

A kit can supply OT-101 as a lyophilized powder in 50-mL glass vials in different quantities, and specify total volume after dissolving (in mL) and resulting concentration (in μM).

A kit can supply OT-101 as a lyophilized powder in 20 mL glass vials in different quantities, and the calculated quantity of OT-101 per patient and treatment cycle can be dissolved in a total volume of 85 ml isotonic saline solution. The CADD ambulatory infusion pump may provide a measured drug therapy to patients in hospital and outpatient settings. It may be used for therapies that require a continuous rate of infusion. Central venous access and the use of luer lock connectors with a split valve septum are recommended when administering drugs via a CADD pump. Drug doses may be concentrated into a small volume. Required materials include a Pump (Smiths Medical CADD SOLIS VIP), Yellow Medication Cassette Reservoirs with Flow Stop, clamp, and female luer 100mL, a CADD Extension Set with male luer, clamp, 0.2 micron air-eliminating filter, and integral antisiphon valve with male luer.

This invention further provides kits comprising a lyophilized powder in a vial at a content of 250 mg each of one or more TGF-β2-specific antisense oligonucleotides selected from SEQ ID NOs: 1-136. The kit may contain the appropriate vial(s) and all necessary components of the application system, i.e., syringes, tube, and filter. OT-101 lyophilized powder can be dissolved in isotonic (0.9%) aqueous sodium chloride prior to use.

### Checkpoint Inhibitor Agents

As referred to herein, checkpoint inhibitors as known in the art are immune checkpoint inhibitor agents. Checkpoint inhibitors are immunotherapy drugs which block checkpoint proteins from binding with their partner proteins. This prevents an “off” signal from being sent, which allows T cells to kill cancer cells. More particularly, checkpoint proteins, such as PD-1 on T cells, keep immune responses in check. Binding of PD-L1 to PD-1 keeps T cells from killing tumor cells. Thus, blocking the binding of PD-L1 to PD-1 with an immune checkpoint inhibitor may allow the T cells to kill tumor cells. The immune system is essentially turned back on so that T cells can attack cancer cells.

In some embodiments, a checkpoint inhibitor of this disclosure may be an inhibitor of CTLA-4, PD-1, or PD-L1.

In certain embodiments, a checkpoint inhibitor of this disclosure may be an inhibitor of PD-1.

In certain embodiments, a checkpoint inhibitor of this disclosure may be pembrolizumab.

In certain embodiments, a checkpoint inhibitor of this disclosure may be pembrolizumab, nivolumab, cemiplimab, spartalizumab, atezolizumab, avelumab, or durvalumab.

Without wishing to be bound by theory, the PD-1 receptor-ligand interaction can be a major pathway hijacked by tumors to suppress immune control. The normal function of PD-1, expressed on the cell surface of activated T cells under healthy conditions, is to down-modulate unwanted or excessive immune responses, including autoimmune reactions. Following T-cell stimulation, PD-1 recruits the tyrosine phosphatases, SHP-1 and SHP-2, to the immunoreceptor tyrosine-based switch motif within its cytoplasmic tail, leading to the dephosphorylation of effector molecules such as CD3 zeta (CD3ζ), protein kinase C-theta (PKCθ), and zeta-chain-associated protein kinase (ZAP70), which are involved in the CD3 T-cell signaling cascade.

### IL-2 Immunotherapeutic Agents

In certain embodiments, an interleukin immunotherapeutic agent of this disclosure may be a natural or synthetic IL-2, a high dose IL-2, a recombinant IL-2, or aldesleukin.

Immunotherapeutic agents can have anti-cancer effects because they may target a tumor microenvironment to activate immune response to cancer cells. For example, interleukin-2 (IL-2) can promote activation of natural killer (NK) cells and cytotoxic CD8⁺ T lymphocytes. Anti-tumor immune response can involve T helper 1 (Th1) and other tumor cell killing activity.

### Anti-Cancer Agents and Therapeutic Combinations

Embodiments of this invention include combinations of TGF-β2 specific inhibitors having none to minimal inhibition of the closely related TGF-β1 and TGF-β3 isoforms, and PD-1 checkpoint inhibitors.

In certain embodiments, this invention provides therapeutic combinations of one or more antisense TGF-β2 inhibitors and a PD-1 checkpoint inhibitor.

Therapeutic embodiments of this invention using one or more agents for inhibiting or suppressing expression of TGF-β2 in combination with a PD-1 immune checkpoint inhibitor agent may be applied for pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

Additional embodiments of this invention include therapeutic combinations of a TGF-β2 inhibitor, an IL-2 immunotherapy agent, and PD-1 checkpoint inhibitor.

In certain embodiments, this invention provides therapeutic combinations of one or more antisense TGF-β2 inhibitors, an IL-2 immunotherapy agent, and a PD-1 checkpoint inhibitor.

Therapeutic embodiments of this invention using one or more agents for inhibiting or suppressing expression of TGF-β2 in combination with a PD-1 immune checkpoint inhibitor agents and an IL-2 immunotherapy agent may be applied for pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

The unexpectedly advantageous synergistic effect of the therapeutic combination for cancer treatment of an antisense TGF-β2 inhibitor, an IL-2 immunotherapy agent, and a checkpoint inhibitor can be markedly increased when the checkpoint inhibitor is a PD-1 checkpoint inhibitor.

In treating cancer patients, use of a therapeutic combination of a PD-1 checkpoint inhibitor, an antisense TGF-β2 inhibitor, and an IL-2 immunotherapy agent can significantly increase overall survival. Overall survival (OS) of such patients can be more than doubled. In some embodiments, therapeutic combination of an antisense TGF-β2 inhibitor, an IL-2 immunotherapy agent, and a PD-1 checkpoint inhibitor can provide an unexpectedly advantageous synergistic effect based on clinical data.

### Synergy of Therapeutic Combinations

Embodiments of this invention can provide synergy for a therapeutic combination of an agent for inhibiting or suppressing expression of TGF-β2 with a PD-1 checkpoint inhibitor and an interleukin immunotherapeutic agent for treating or ameliorating the symptoms of cancer.

In some embodiments, the anti-cancer use of the combination of an antisense agent for inhibiting or suppressing expression of TGF-β2, a PD-1 checkpoint inhibitor, and an IL-2 immunotherapeutic agent can be particularly effective for patients with high levels of tumor associated monocytes and/or tumor associated macrophages. Thus, high levels of tumor associated monocytes and/or tumor associated macrophages can be used as biomarkers to select patients who will benefit from the combination therapy. This synergistic effect may be strongest for the combination therapy with a PD-1 checkpoint inhibitor, as compared to CTLA4 or PD-L1-specific checkpoint inhibitors.

Without wishing to be bound by theory, because PD-1 is present in M2-type tumor associated macrophages, and antisense agents for inhibiting or suppressing expression of TGF-β2 have effects in repolarizing M2 and promoting anti-tumor effects, these two agents appear to be operable synergistically against the same target when used in combination with IL-2. TGF-β2 may have a central role in programming M1-type tumor associated macrophages, which can exhibit anti-tumor effects. In some aspects of this invention, inhibiting or suppressing TGF-β2 with antisense agents can have anti-tumor effects. The antisense agents may have the effect of re-programming to promote M1-type tumor associated macrophages. Such re-programming may have the effect of actively reducing and/or eliminating cancer tumors, especially when combined with an agent that is hampered by high TGF-β2.

For a valid stratification without any bias, the median of the population data is defined as the cutoff. For each gene of interest, the median expression level across all the samples is calculated. The median is the middle value in a list of numbers sorted in ascending or descending order and is used because it is less affected by outliers than the mean. Samples are then stratified into two groups based on whether the expression level of a particular gene is above or below the median. This creates a “high expression” group and a “low expression” group.

As used herein, an “immunogenically hot tumor” refers to a type of cancer that elicits a strong response from the patient's immune system. Conversely, “cold” tumors have low immunogenicity, meaning they do not provoke a strong immune response.

In some embodiments, the agents, uses or methods of this invention can be applied to an immunogenically cold pancreatic cancer, or an immunogenically hot melanoma.

In some embodiments, the agents, uses or methods of this invention can be applied to cancers that are in between immunogenically cold and immunogenically hot, which can be a skin cancer, a lung cancer, a breast cancer, a prostate cancer, a colorectal cancer, a kidney cancer, a stomach cancer, an ovarian cancer, a cervical cancer, a liver cancer, or a multiple myeloma.

Numbered embodiments of this invention include the following:
1) An antisense agent for inhibiting or suppressing expression of TGF-β2 in combination with a checkpoint inhibitor agent for use in treating or ameliorating the symptoms of cancer in a human subject or animal.
2) Use of an antisense agent for inhibiting or suppressing expression of TGF-β2 in the preparation of a medicament for treating or ameliorating the symptoms of a cancer in a human subject or animal in combination with a checkpoint inhibitor agent.
3) A method for treating or ameliorating the symptoms of cancer in a human or animal subject in need, the method comprising:
   administering a therapeutically effective amount of an antisense agent for inhibiting or suppressing expression of TGF-β2 to the subject;
   administering a therapeutically effective amount of a checkpoint inhibitor agent to the subject.
4) The agent of embodiment 1, in combination with an interleukin immunotherapeutic agent.
5) The use of embodiment 2, in combination with an interleukin immunotherapeutic agent.
6) The method of embodiment 3, comprising administering a therapeutically effective amount of an interleukin immunotherapeutic agent to the subject.

7) The agent, use or method of any of embodiments 1-6, wherein the agent for inhibiting or suppressing expression of TGF-β2, the checkpoint inhibitor, and the interleukin immunotherapeutic agent are administered concurrently, simultaneously, sequentially, or separately in time.
8) The agent, use or method of any of embodiments 1-7, wherein the agent for inhibiting or suppressing expression of TGF-β2, the checkpoint inhibitor, and the interleukin immunotherapeutic agent are administered separately or in combined formulation by injection or infusion.
9) The agent, use or method of any of embodiments 1-8, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.
10) The agent, use or method of any of embodiments 1-9, wherein the agent for inhibiting or suppressing expression of TGF-β2 is a TGF-β2-specific antisense oligonucleotide complementary to a TGF-β2 transcript and 15-30 nucleotides in length.
11) The agent, use or method of any of embodiments 1-10, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.
12) The agent, use or method of any of embodiments 1-11, wherein the agent for inhibiting or suppressing expression of TGF-β2 is one or more TGF-β2-specific antisense oligonucleotides as shown in Table 1, complementary to a TGF-β2 transcript, and chemically-modified variants thereof, LNA variants thereof, gapmer variants thereof, and any combination or pooling thereof.
13) The agent, use or method of any of embodiments 1-12, wherein the TGF-β2-specific antisense oligonucleotides have no more than one or two mismatches as compared to a target human TGF-β2.
14) The agent, use or method of any of embodiments 1-13, wherein the TGF-β2-specific antisense oligonucleotides reduce a TGF-β2 transcript level by at least 60%, or at least 70%, or at least 80%, or at least 90%.
15) The agent, use or method of any of embodiments 1-14, wherein the TGF-β2-specific antisense oligonucleotides reduce any TGF-β1 transcript level and any TGF-β3 transcript level by less than 10%, or less than 5%, or less than 1%.
16) The agent, use or method of any of embodiments 1-15, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
17) The agent, use or method of any of embodiments 1-16, wherein the antisense agent is conjugated to a polyethylene glycol, a lipid, or a triantenarry N-acteyl-galactosamine.
18) The agent, use or method of any of embodiments 1-17, wherein each agent comprises a carrier of sterile water for injection, saline, isotonic saline, or a combination thereof, which may be the same or different for each agent.
19) The agent, use or method of any of embodiments 1-18, wherein the agents are substantially free of excipients.
20) The agent, use or method of any of embodiments 1-19, wherein the agents are stable in a carrier substantially free of excipients for at least 14 days at 37° C.
21) The agent, use or method of any of embodiments 1-20, wherein the checkpoint inhibitor agent is an inhibitor of PD-1.
22) The agent, use or method of any of embodiments 1-21, wherein the checkpoint inhibitor agent is pembrolizumab, nivolumab, cemiplimab, spartalizumab, atezolizumab, avelumab, or durvalumab.
23) The agent, use or method of any of embodiments 1-22, wherein the interleukin immunotherapeutic agent is a natural IL-2, a high dose IL-2, a recombinant IL-2, or aldesleukin.
24) The agent, use or method of any of embodiments 1-23, comprising selecting subjects who benefit from the agent, use or method based on levels of one or more biomarkers TGF-β2, IL-2, CD19, IRF5, ITGAM, and a combination thereof.
25) The agent, use or method of any of embodiments 1-24, wherein the one or more biomarkers is IRF5 and the subject is selected when expression of IRF5 is at a level above the median.
26) The agent, use or method of any of embodiments 1-25, wherein the one or more biomarkers is ITGAM and the subject is selected when expression of ITGAM is at a level above the median.
27) The agent, use or method of any of embodiments 1-26, wherein the subject after the administration or use has a decreased level of TGF-β2 as compared to before the administration or use.
28) The agent, use or method of any of embodiments 1-27, wherein the subject after the administration or use has an increased level of IRF5 as compared to before the administration or use.
29) The agent, use or method of any of embodiments 1-28, wherein the subject after the administration or use has a decreased level of ITGAM as compared to before the administration or use.
30) The agent, use or method of any of embodiments 1-29, comprising administering a therapeutically effective amount of an expression product of IRF5 or ITGAM to the subject.
31) The agent, use or method of any of embodiments 1-30, wherein the expression product is an mRNA, polypeptide, protein, or fragment thereof, or combination thereof.
32) The agent, use or method of any of embodiments 1-31, wherein the administration or use decreases mortality rate at month 6, 12, 18, 24, 30, or 36.
33) The agent, use or method of any of embodiments 1-32, wherein the administration or use increases overall survival rate at month 6, 12, 18, 24, 30, or 36.
34) The agent, use or method of any of embodiments 1-33, in combination with any one or more medicaments comprising a targeted cancer drug, a cancer growth blocker, an EGFR inhibitor, and combinations thereof.
35) The agent, use or method of any of embodiments 1-34, in combination with any one or more medicaments selected from bevacizumab, everolimus, belzutifan, dabrafenib, trametinib, and combinations thereof.
36) The agent, use or method of any of embodiments 1-35, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.
37) The agent, use or method of any of embodiments 1-36, in combination with any one or more medicaments which are EGFR inhibitors selected from erlotinib, gefitinib, afatinib, osimertinib, dacomitininb, and combinations thereof.
38) The agent, use or method of any of embodiments 1-37, in combination with a chemotherapy medicament.
39) The agent, use or method of any of embodiments 1-38, in combination with radiation therapy or electric field therapy.
40) An antisense agent for inhibiting or suppressing expression of TGF-β2 in combination with an interleukin immunotherapeutic agent for use in treating or ameliorating the symptoms of cancer in a human subject or animal.
41) Use of an antisense agent for inhibiting or suppressing expression of TGF-β2 in the preparation of a medicament for treating or ameliorating the symptoms of a cancer in a human subject or animal in combination with an interleukin immunotherapeutic agent.
42) A method for treating or ameliorating the symptoms of cancer in a human or animal subject in need, the method comprising:
   administering a therapeutically effective amount of an antisense agent for inhibiting or suppressing expression of TGF-β2 to the subject;
   administering a therapeutically effective amount of an interleukin immunotherapeutic agent to the subject.
43) The agent, use or method of any of embodiments 40-42, wherein the agent for inhibiting or suppressing expression of TGF-β2, and the interleukin immunotherapeutic agent are administered concurrently, simultaneously, sequentially, or separately in time.
44) The agent, use or method of any of embodiments 40-43, wherein the agent for inhibiting or suppressing expression of TGF-β2, and the interleukin immunotherapeutic agent are administered separately or in combined formulation by injection or infusion.
45) The agent, use or method of any of embodiments 40-44, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.
46) The agent, use or method of any of embodiments 40-45, wherein the agent for inhibiting or suppressing expression of TGF-β2 is a TGF-β2-specific antisense oligonucleotide complementary to a TGF-β2 transcript and 15-30 nucleotides in length.
47) The agent, use or method of any of embodiments 40-46, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.
48) The agent, use or method of any of embodiments 40-47, wherein the agent for inhibiting or suppressing expression of TGF-β2 is one or more TGF-β2-specific antisense oligonucleotides as shown in Table 1, complementary to a TGF-β2 transcript.
49) The agent, use or method of any of embodiments 40-48, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
50) The agent, use or method of any of embodiments 40-49, wherein the antisense agent is conjugated to a polyethylene glycol, a lipid, or a triantenarry N-acteyl-galactosamine.
51) The agent, use or method of any of embodiments 40-50, wherein each agent comprises a carrier of sterile water for injection, saline, isotonic saline, or a combination thereof, which may be the same or different for each agent.
52) The agent, use or method of any of embodiments 40-51, wherein the agents are substantially free of excipients.
53) The agent, use or method of any of embodiments 40-52, wherein the agents are stable in a carrier substantially free of excipients for at least 14 days at 37° C.

54) The agent, use or method of any of embodiments 40-53, wherein the interleukin immunotherapeutic agent is a natural IL-2, a high dose IL-2, a recombinant IL-2, or aldesleukin.
55) The agent, use or method of any of embodiments 40-54, wherein the administration or use decreases mortality rate at month 6, 12, 18, 24, 30, or 36.
56) The agent, use or method of any of embodiments 40-55, wherein the administration or use increases survival rate at month 6, 12, 18, 24, 30, or 36.
57) The agent, use or method of any of embodiments 40-56, in combination with any one or more medicaments comprising a targeted cancer drug, a cancer growth blocker, an EGFR inhibitor, and combinations thereof.
58) The agent, use or method of any of embodiments 40-57, in combination with any one or more medicaments selected from bevacizumab, everolimus, belzutifan, dabrafenib, trametinib, and combinations thereof.
59) The agent, use or method of any of embodiments 40-58, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.
60) The agent, use or method of any of embodiments 40-59, in combination with any one or more medicaments which are EGFR inhibitors selected from erlotinib, gefitinib, afatinib, osimertinib, dacomitininb, and combinations thereof.
61) The agent, use or method of any of embodiments 40-60, in combination with a chemotherapy medicament.
62) The agent, use or method of any of embodiments 40-61, in combination with radiation therapy or electric field therapy.
63) The agent, use or method of any of embodiments 40-62, comprising selecting subjects who benefit from the agent, use or method based on levels of one or more biomarkers TGF-β2, IRF5, ITGAM, and a combination thereof.
64) The agent, use or method of any of embodiments 40-63, comprising selecting subjects who benefit from the agent, use or method based on levels of one or more biomarkers TGF-β2, IRF5, ITGAM, neoantigen, mutational load, macrophage and a combination thereof.
65) The agent, use or method of any of embodiments 40-64, wherein the one or more biomarkers is IRF5 and the subject is selected when expression of IRF5 is at a level below the median.
66) The agent, use or method of any of embodiments 40-65, wherein the one or more biomarkers is tumor associated macrophage and the subject is selected when tumor associated macrophage is below average.
67) The agent, use or method of any of embodiments 40-66, wherein the one or more biomarkers is tumor neoantigen mutation load and the subject is selected when neoantigen tumor load is below average.
68) The agent, use or method of any of embodiments 40-67, comprising administering a therapeutically effective amount of an agent for inhibiting or suppressing expression of ITGAM or IRF5 to the subject.
69) The agent, use or method of any of embodiments 40-68, wherein the agent for inhibiting or suppressing expression of ITGAM or IRF5 is an antisense oligonucleotide targeted to ITGAM or IRF5, respectively.
70) A kit for treating or ameliorating the symptoms of cancer, the kit comprising:
   a therapeutically effective amount of an antisense agent for inhibiting or suppressing expression of TGF-β2; and
   a therapeutically effective amount of a checkpoint inhibitor agent.
71) The kit of embodiment 70, comprising a therapeutically effective amount of an interleukin immunotherapeutic agent.
72) The kit of any of embodiments 70-71, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, a thymus cancer, or a multiple myeloma.
73) The kit of any of embodiments 70-72, wherein the agent for inhibiting or suppressing expression of TGF-β2 is a TGF-β2-specific antisense oligonucleotide complementary to a TGF-β2 transcript and 15-30 nucleotides in length.
74) The kit of any of embodiments 70-73, wherein the agent for inhibiting or suppressing expression of TGF-β2 is a TGF-2-specific antisense oligonucleotides complementary to a TGF-β2 pre-RNA, pre-mRNA or mRNA and 18-21 nucleotides in length.
75) The kit of any of embodiments 70-74, wherein the agent for inhibiting or suppressing expression of TGF-β2 is one or more TGF-β2-specific antisense oligonucleotides as shown in Table 1, complementary to a TGF-β2 transcript.
76) The kit of any of embodiments 70-75, wherein the TGF-β2-specific antisense oligonucleotides have no more than one or two mismatches as compared to a target human TGF-β2.
77) The kit of any of embodiments 70-76, wherein the TGF-β2-specific antisense oligonucleotides reduce a TGF-β2 transcript level by at least 60%, or at least 70%, or at least 80%, or at least 90%.
78) The kit of any of embodiments 70-77, wherein the TGF-β2-specific antisense oligonucleotides reduce any TGF-β1 transcript level and any TGF-β3 transcript level by less than 10%, or less than 5%, or less than 1%.
79) The kit of any of embodiments 70-78, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
80) The kit of any of embodiments 70-79, wherein the antisense agent is conjugated to a polyethylene glycol, a lipid, or a triantenarry N-acteyl-galactosamine.
81) The kit of any of embodiments 70-80, wherein each agent comprises a carrier of sterile water for injection, saline, isotonic saline, or a combination thereof, which may be the same or different for each agent.
82) The kit of any of embodiments 70-81, wherein the agents are substantially free of excipients.
83) The kit of any of embodiments 70-82, wherein the agents are stable in a carrier substantially free of excipients for at least 14 days at 37° C.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

It is emphasized herein according to common practice the features of the drawings have arbitrary scale and are intended to cover similar features that may be arbitrarily expanded or reduced.

### EXAMPLES

Example 1. A clinical study was performed for understanding overall survival of melanoma patients for a therapeutic combination of an antisense TGF-β2 inhibitor, an IL-2 immunotherapy agent, and a PD-1 checkpoint inhibitor. Clinical results were obtained for 423 patients having melanoma cancer diagnosis (Sec KM plotter for immunotherapy, available at https://kmplot.com/analysis/index.php?p=service&cancer=immunotherapy).

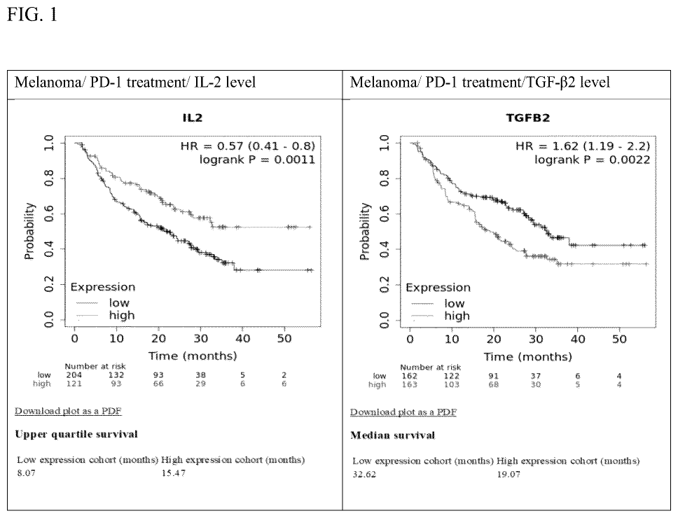

FIG. 1 shows the results of this clinical study of overall survival of melanoma patients. FIG. 1 shows Kaplan-Meier overall survival charts for a study of such clinical effects. FIG. 1 shows that for use of a PD-1 inhibitor, improved survival was indicated for high IL2 (left panel), which can be provided with an IL-2 immunotherapy agent, and low TGF-β2 (right panel), which can be provided with an antisense TGF-β2 inhibitor. These clinical data showed basis for a therapeutic combination of an antisense TGF-β2 inhibitor, a PD-1 checkpoint inhibitor, and an IL-2 immunotherapy agent for improved overall survival of melanoma patients. Logrank P values in FIG. 1 indicate high significance for improved survival using this therapeutic combination based on this clinical study.

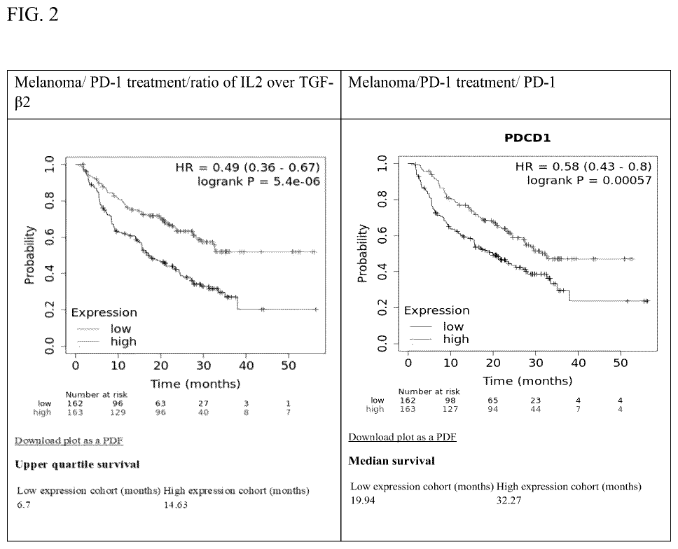

FIG. 2 shows the results of a clinical study on overall survival of melanoma patients. FIG. 2 shows Kaplan-Meier overall survival charts for a study of such clinical effects. FIG. 2 shows that for use of a PD-1 inhibitor, improved survival was highly indicated for a higher ratio of IL-2/TGF-β2 (left panel), which can be provided with an IL-2 immunotherapy agent and an antisense TGF-β2 inhibitor. These clinical data showed basis for a therapeutic combination of an antisense TGF-β2 inhibitor, a PD-1 checkpoint inhibitor, and an IL-2 immunotherapy agent for improved overall survival of melanoma patients. The Logrank P value in FIG. 2 (left panel) indicates high significance for improved survival using this therapeutic combination based on this clinical study.

FIG. 2 clinical data showed that a high IL-2/TGF-β2 ratio, corresponding to high IL2 and low TGF-β2, was a strong driver of survival for melanoma patients. With the high IL-2/TGF-β2 ratio, survival in the upper quartile of melanoma patients was surprisingly elevated from 6.7 to 15 months (p=5.4e-06) (FIG. 2, left panel). Thus, these clinical data showed basis for a surprising synergistic effect of the therapeutic combination of an antisense TGF-β2 inhibitor, a PD-1 checkpoint inhibitor, and an IL-2 immunotherapy agent for improved overall survival of melanoma patients.

Example 2. A clinical study was performed for understanding overall survival of melanoma (N=162) and pancreatic cancer (N=88) patients with a therapeutic combination of an antisense TGF-β2 inhibitor, an IL-2 immunotherapy agent, and a PD-1 checkpoint inhibitor. (See KM plotter for immunotherapy, available at https://kmplot.com/analysis/index.php?p=service&cancer=immunotherapy) and pancreatic cancer (N=177) (KM Plotter Pan-cancer RNA-seq, available at https://kmplot.com/analysis/index.php?p=service&cancer=pancancer_rnaseq).

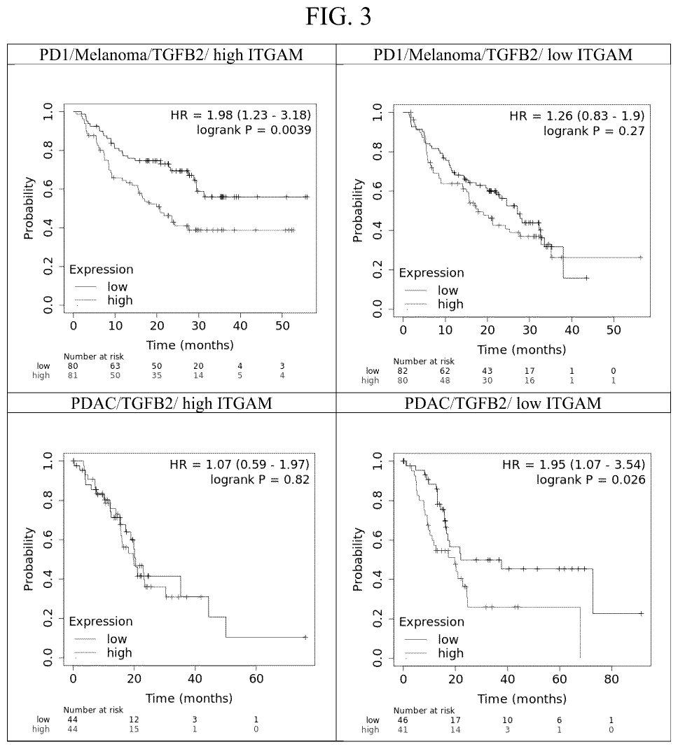

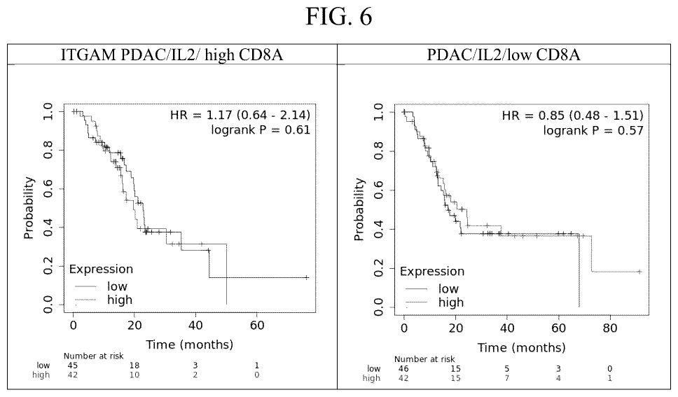

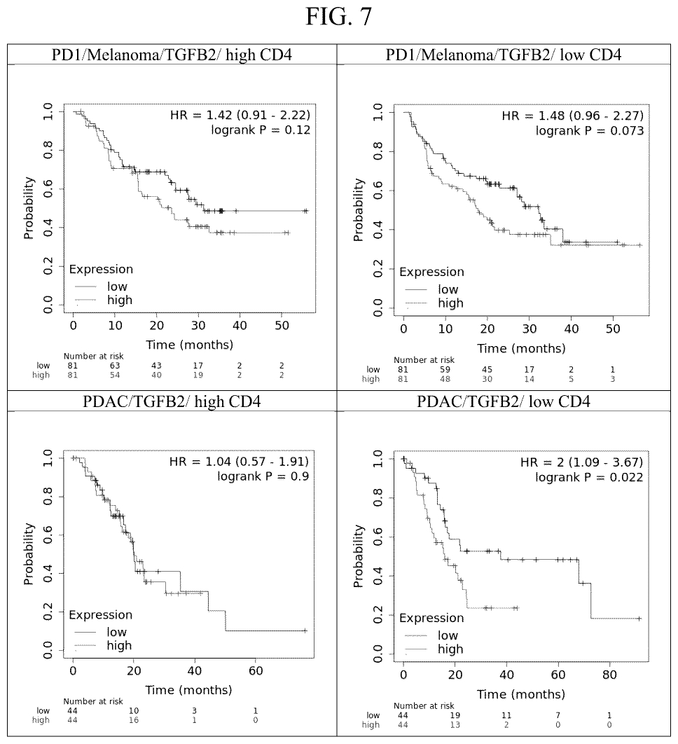

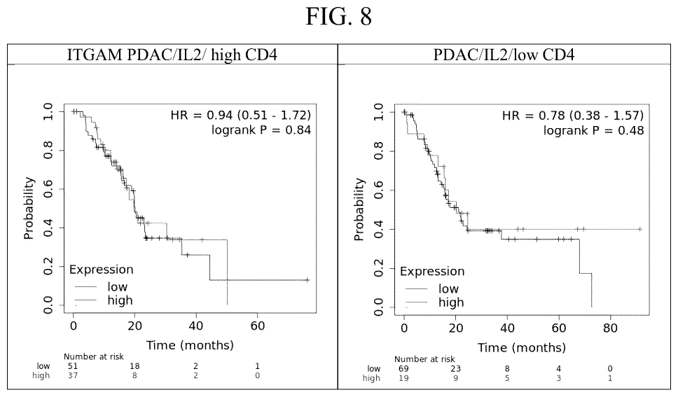

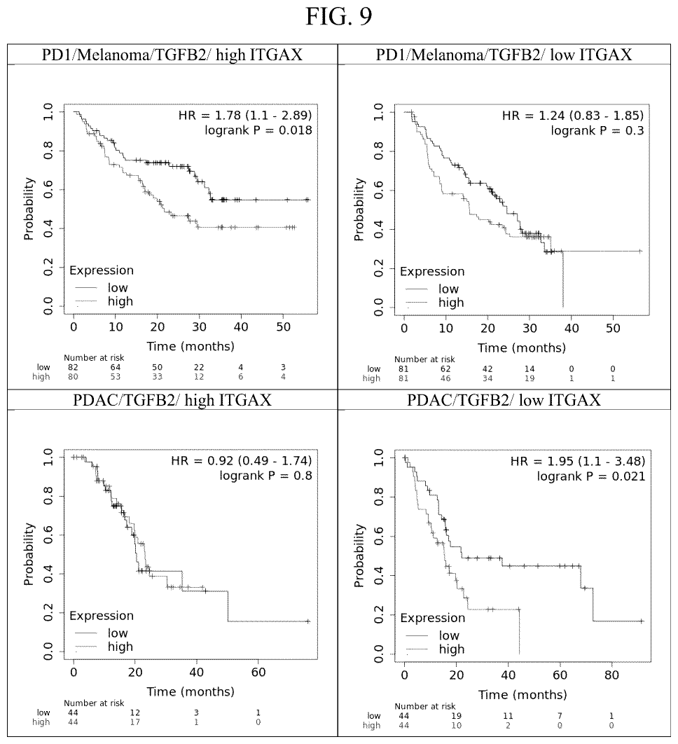

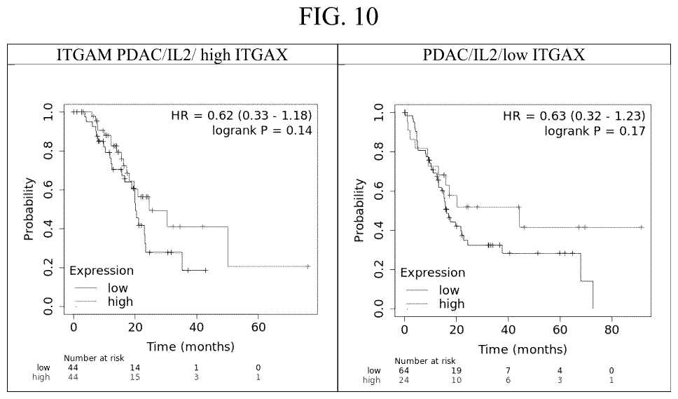

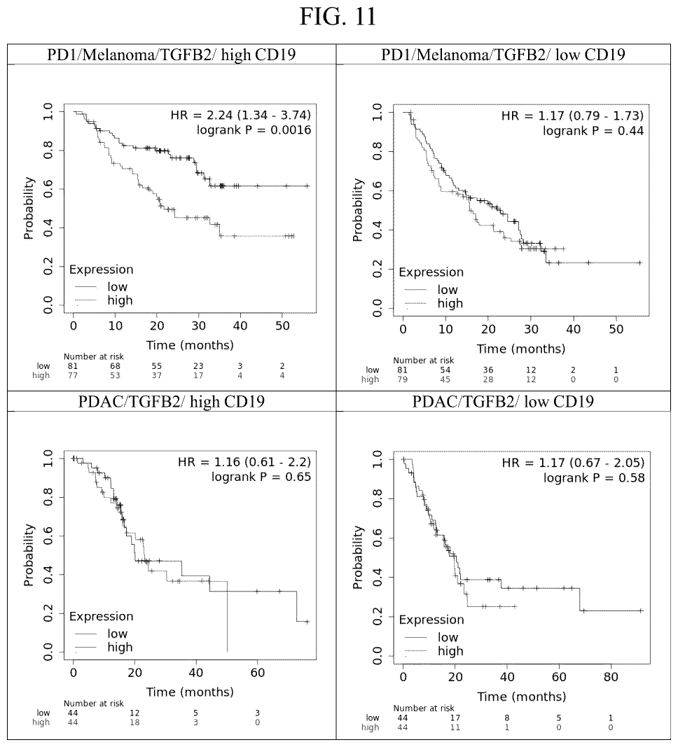

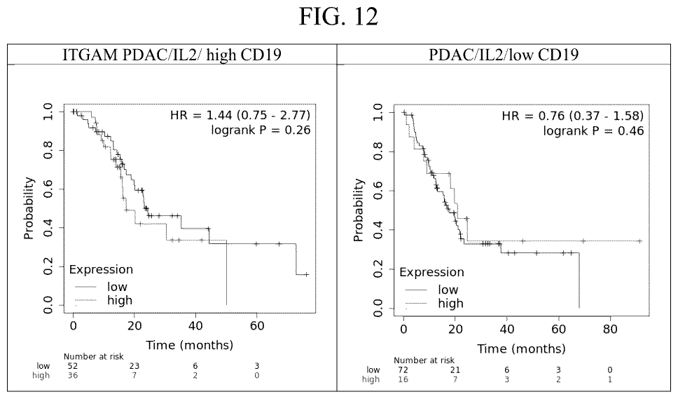

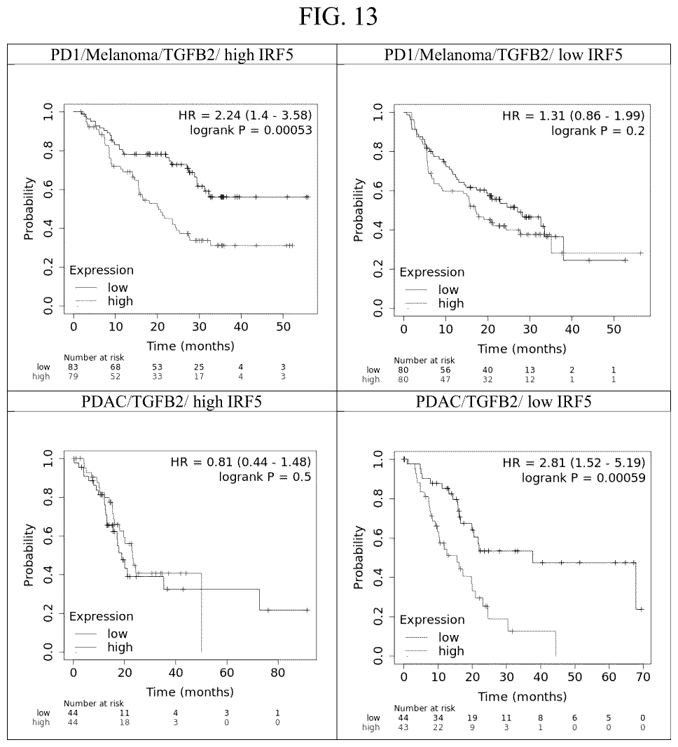

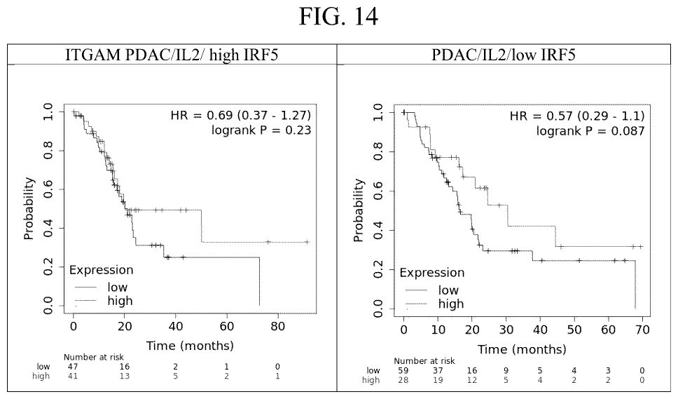

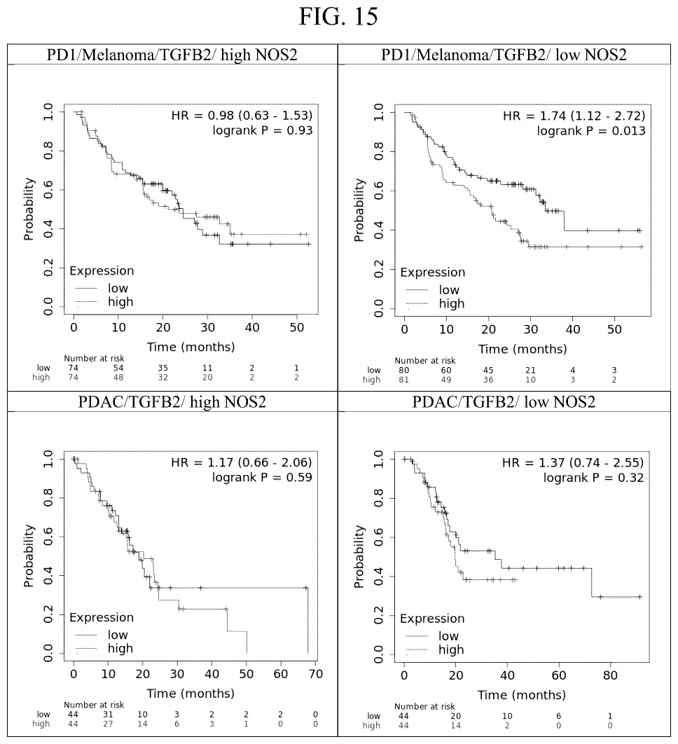

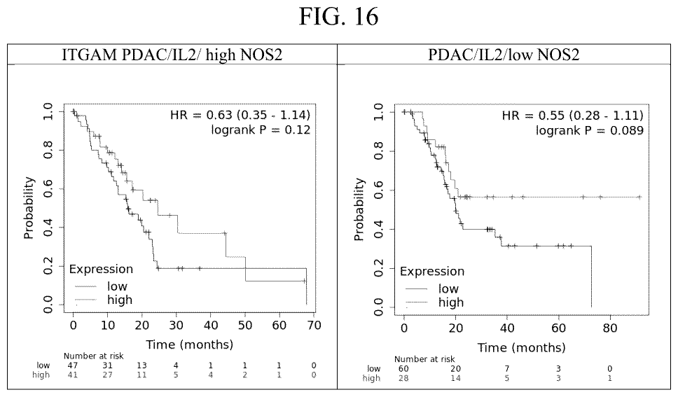

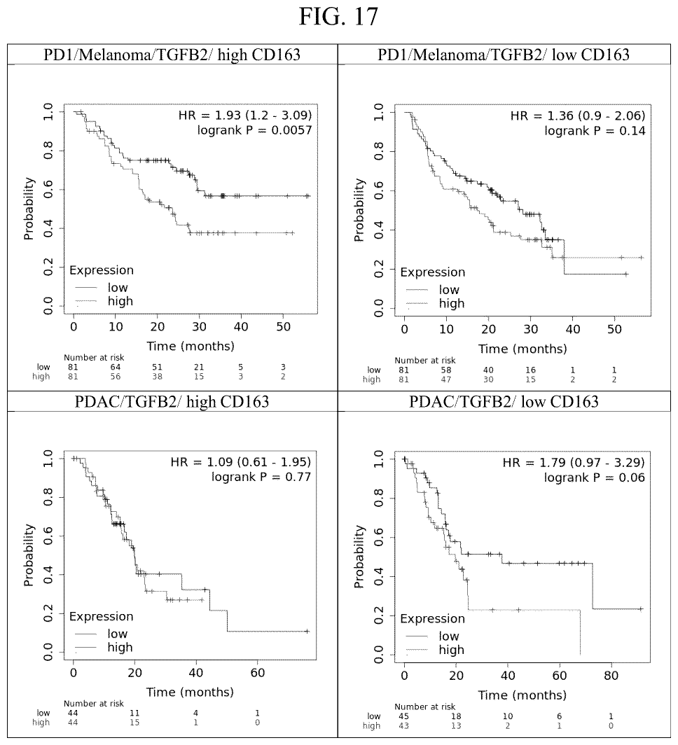

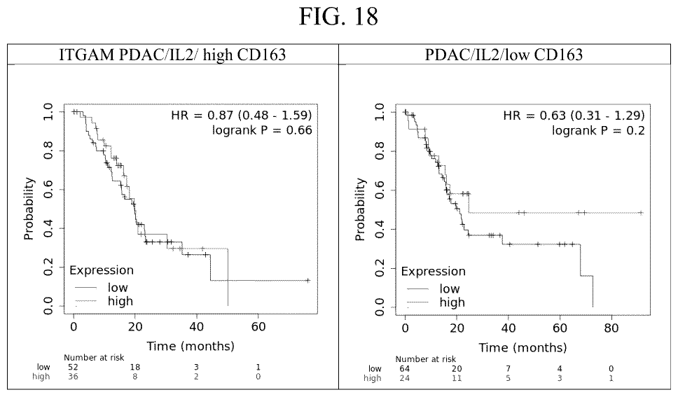

FIGS. 3-18 show the results of this clinical study of overall survival of melanoma patients treated with PD-1 therapeutics and pancreatic cancer patients not treated. FIGS. 3-18 show Kaplan-Meier overall survival charts for a study of such clinical effects, covering two extremes: hot immunogenic melanoma tumors and cold nonimmunogenic pancreatic tumors.

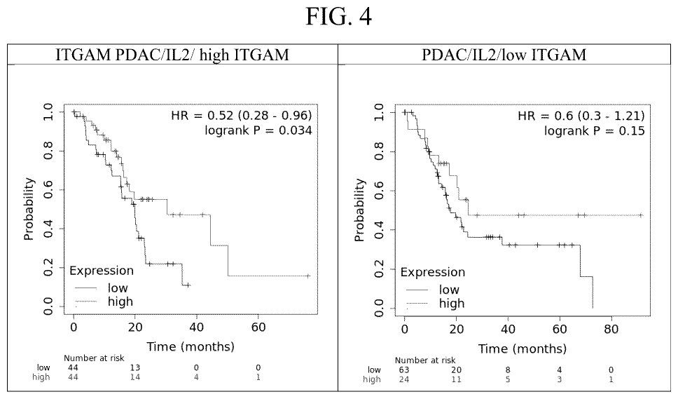

FIGS. 3, 5, 7, 9, 11, 13, 15 and 17 (upper left, right panels) show Kaplan-Meier overall survival charts for this study of melanoma patients. FIGS. 3, 5, 7, 9, 11, 13, 15 and 17 (lower left, right panels) show Kaplan-Meier overall survival charts for this study of pancreatic cancer patients (PDAC) through the lens of TGF-β2 expression. FIGS. 4, 6, 8, 10, 12, 14, 16 and 18 (left, right panels) show Kaplan-Meier overall survival charts for this study of pancreatic cancer patients (PDAC) through the lens of IL-2 expression.

FIG. 3 (upper left panel) shows that for use of a PD-1 inhibitor in melanoma patients, improved survival was indicated for low TGF-β2 and high ITGAM. These data show that high ITGAM was a useful biomarker of overall survival of patients with a therapeutic combination of an antisense TGF-β2 inhibitor and a PD-1 checkpoint inhibitor, and can be used to select patients that would benefit. FIG. 3 (upper right panel) shows that low ITGAM would not be a preferred indicator under those conditions.

FIG. 3 (lower left, right panels) shows that for use of a PD-1 inhibitor in pancreatic cancer patients (PDAC), neither low nor high ITGAM would be a preferred indicator.

FIG. 4 (left panel) shows that for use of a PD-1 inhibitor in pancreatic cancer patients (PDAC), improved survival was indicated for low TGF-β2 and high ITGAM. The value Logrank P=0.034 indicates significance (<0.05), although even lower P values are preferred. These data show that high ITGAM was a useful biomarker of overall survival of patients with a therapeutic combination of an antisense TGF-β2 inhibitor and a PD-1 checkpoint inhibitor, and can be used to select patients that would benefit. FIG. 4 (right panel) shows that low ITGAM would not be a preferred indicator under those conditions.

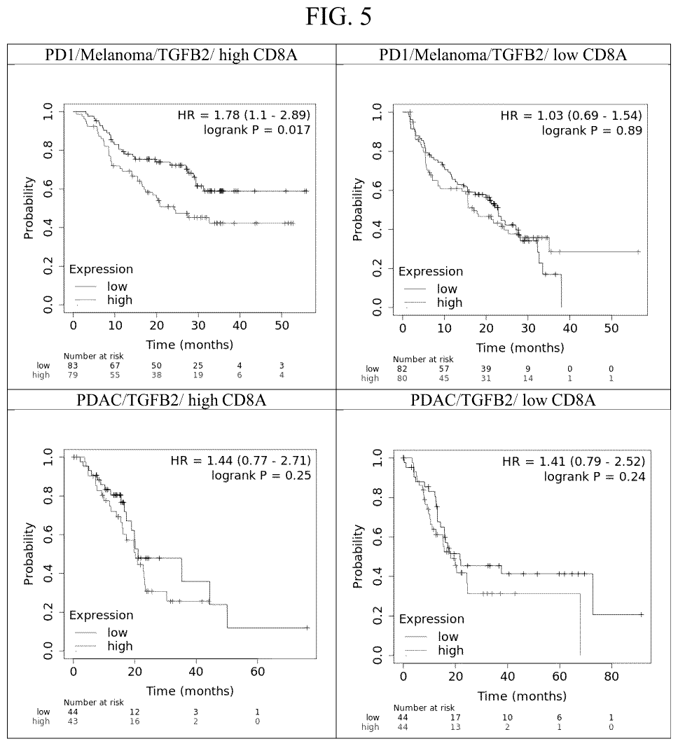

FIG. 5 (upper left panel) shows that for use of a PD-1 inhibitor in melanoma patients, neither low nor high CD8A would be a preferred indicator.

FIG. 5 (lower left, right panels) shows that for use of a PD-1 inhibitor in pancreatic cancer patients (PDAC), neither low nor high CD8A would be a preferred indicator.

By similar analysis of data of FIGS. 3-18, conclusions are summarized in Table 3.

TABLE 3
Biomarkers of preferred significance for melanoma and PDAC

| | Biomarkers of preferred | |
|---|---|---|
| | significance for | |
| Cancer (stratifier) | enhanced stratification | FIGS. |
| Melanoma (TGF-β2) | ITGAM, CD19, IRF5, CD163 | 3-4, 11-12, 13-14, |
| | | 17-18 |
| PDAC (TGF-β2) | IRF5 (M1 macrophage) | 13-14 |
| PDAC (IL-2) | ITGAM (neutrophils) | 3-4 |

The data can be summarized for melanoma treated with PD-1 as follows: CD19 and IRF5 were highly significant for outcome improvement in TGF-β2 driven overall survival as compared to melanoma treated with PD-1 alone.

The data can be summarized for PDAC as follows: IRF5 low and CD19 low were significant for outcome improvement in TGF-β2 driven overall survival. The impact of these biomarkers on PDAC for IL-2 driven overall survival was not significant except for ITGAM.

Thus, the first finding of this clinical study was that ITGAM, CD19, IRF5, and CD163 were useful biomarkers of overall survival of melanoma patients with a therapeutic combination of an antisense TGF-β2 inhibitor and a PD-1 checkpoint inhibitor.

A second finding of this clinical study was that IRF5 was a useful biomarker of overall survival of pancreatic cancer patients (PDAC) with a therapeutic antisense TGF-β2 specific inhibitor.

A third finding of this clinical study was that ITGAM may be a useful biomarker of overall survival of pancreatic cancer patients (PDAC) with a therapeutic combination of an IL-2 therapy and an PD-1 checkpoint inhibitor.

Another finding of this clinical study was that IRF5 was particularly useful as a biomarker of overall survival of melanoma patients with a therapeutic antisense TGF-β2 specific inhibitor.

Another finding of this clinical study was that IRF5 was particularly useful as a biomarker of overall survival of pancreatic cancer patients (PDAC) with a therapeutic combination of an antisense TGF-β2 inhibitor and a PD-1 checkpoint inhibitor.

This clinical study relates to the following cell types:
B cells=CD19
CD8⁺ T cell=CD8A
CD4⁺ T cell=CD4
M1 macrophage=NOS2 or IRF5
M2 macrophage=CD163
Neutrophil=ITGAM
Dendritic cell=ITGAX

Example 3. A clinical study was performed for understanding overall survival of pancreatic cancer (PDAC) patients with a therapeutic combination of an antisense TGF-β2 inhibitor, an IL-2 immunotherapy agent, and a PD-1 checkpoint inhibitor.

The study was performed for understanding overall survival of melanoma treated with immunotherapy (N=423) (See KM plotter for immunotherapy, available at https://kmplot.com/analysis/index.php?p=service&cancer=immunotherapy) and pancreatic cancer (N=177) (KM Plotter Pan-cancer RNA-seq, https://kmplot.com/analysis/index.php?p=service&cancer=pancancer_rnaseq).

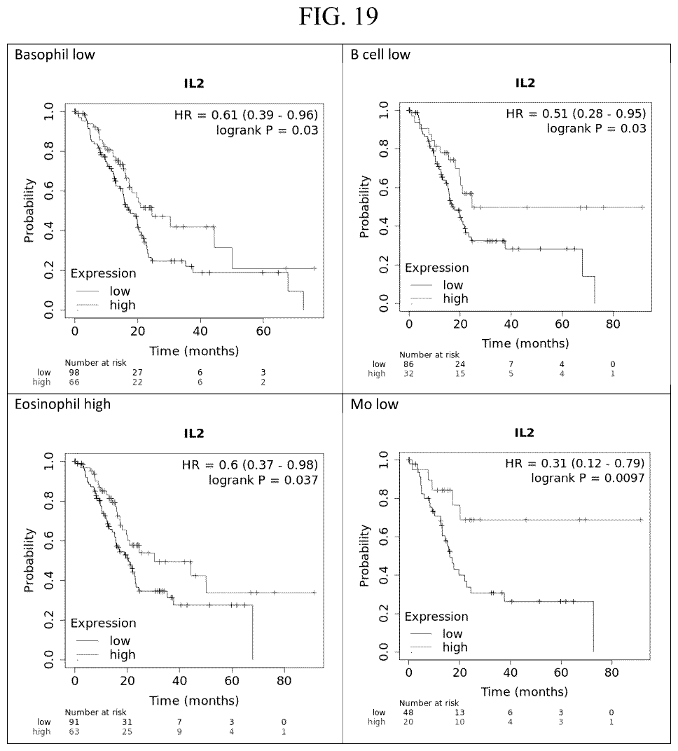

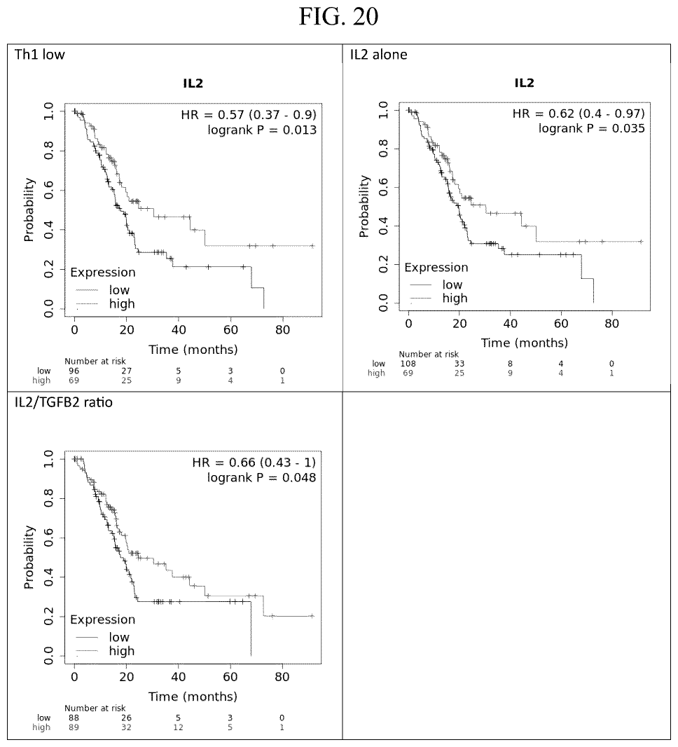

FIGS. 19-20 show the results of this clinical study of overall survival of PDAC patients. FIGS. 19-20 show Kaplan-Meier overall survival charts for a study of such clinical effects.

FIGS. 19-20 shows that for IL-2 driven survival, with additional stratification based on various immune cell indicators, namely basophils, B cells, eosinophils, and M0 macrophages, and Th1 helper cells, a high level of IL-2 significantly increased survival in all cases. M0 macrophages were a highly significant factor. FIGS. 19-20 show that Logrank P values indicated significance for improved survival using an IL-2 immunotherapy agent in combination with a PD-1 checkpoint inhibitor based on this clinical study and conditions. FIG. 20 (upper right panel and lower panel) showed comparative baseline results.

This clinical study of pancreatic cancer (PDAC) showed a synergistic effect of low macrophage on an IL-2 immunotherapy in PDAC.

Example 4. A clinical study was performed for understanding overall survival of pancreatic cancer patients with a therapeutic antisense TGF-β2 inhibitor. Clinical results were obtained for 80-108 patients having pancreatic cancer diagnosis (cBioPortal For Cancer Genomics).

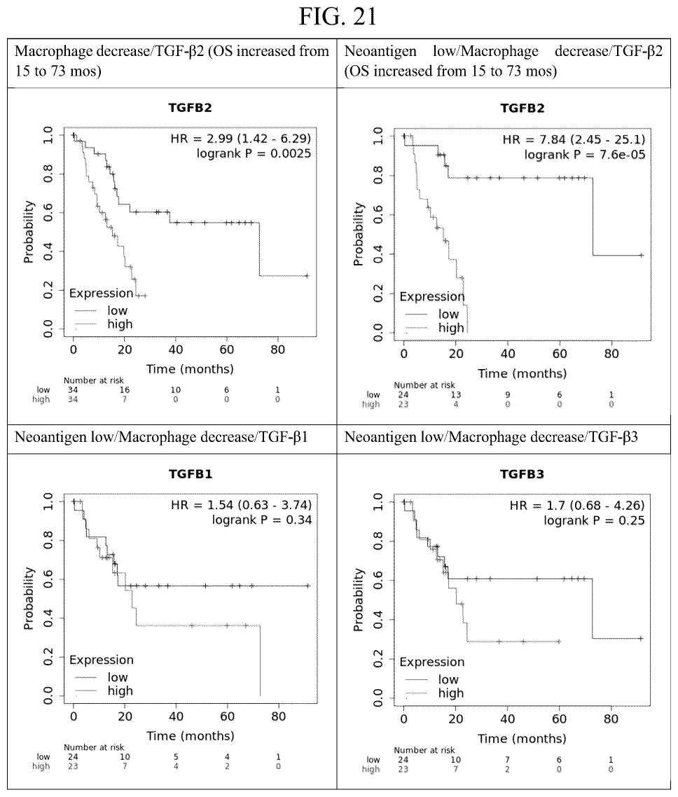

FIG. 21 shows the results of this clinical study of overall survival of pancreatic cancer patients. FIG. 21 shows Kaplan-Meier overall survival charts for a study of such clinical effects. FIG. 21 (upper left panel) shows that for patients with low tumor-associated-macrophage, a high tumoral mRNA level of TGF-β2 significantly decreased survival. Logrank P value in FIG. 21 (upper left panel) indicates high significance for improved survival using a therapeutic antisense TGF-β2 inhibitor based on this clinical study and conditions. Survival of patients in the high range of tumoral TGF-β2 expression was only 15 months, as compared to 73 months for patients in the low range of tumoral TGF-β2 expression.

FIG. 21 (upper right panel) shows that for patients with low tumor-associated-macrophage and low mutational burden (neoantigen), a high tumoral mRNA level of TGF-β2 significantly decreased survival. Logrank P value in FIG. 21 (upper right panel) indicates high significance for improved survival using a therapeutic antisense TGF-β2 inhibitor based on this clinical study and conditions. Survival of patients in the high range of tumoral TGF-β2 expression was only 15 months, as compared to 73 months for patients in the low range of tumoral TGF-β2 expression. Only TGF-β2 was shown to have impact on survival. There was no impact on survival with either TGF-β1 or TGF-β3 (See Logrank P, FIG. 21 (lower left and right panels, respectively)).

Thus, this clinical study showed that tumoral mRNA level of TGF-β2, low tumor-associated-macrophage, and low mutational burden (neoantigen) were biomarkers for improved overall survival of pancreatic cancer patients with therapeutic antisense TGF-β2 inhibitors. These biomarkers can be used for selecting patients who benefit from TGF-β2 inhibitor treatment.

Example 5. A clinical study was performed for understanding overall survival of cancer patients with a therapeutic combination of an antisense TGF-β2 inhibitor, an IL-2 immunotherapy agent, and a checkpoint inhibitor. Clinical results were obtained for 1,045 patients having cancer diagnosis (cBioPortal For Cancer Genomics). Clinical results were obtained for a range of cancer diagnoses including Bladder (N=73), Esophageal adenocarcinoma (N=103), Glioblastoma (N=28), Hepatocellular carcinoma (N=22), HNSCC (N=5), Melanoma (N=423), NSCLC (N=21), NSCL (N=22), and Urothelial (N=348).

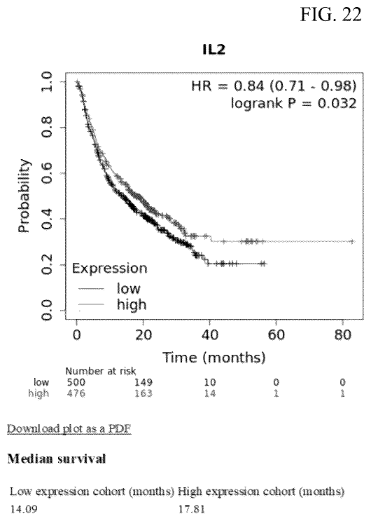

FIGS. 22-30 show the results of this clinical study of overall survival of cancer patients. FIGS. 22-30 show Kaplan-Meier overall survival charts for a study of such clinical effects.

FIG. 22 shows that for use of any checkpoint inhibitor (PD-1, PD-L1, or CTLA-4), across all tumor types, a high level of IL-2 significantly increased survival. Logrank P value in FIG. 22 indicates significance for improved survival using an IL-2 immunotherapy agent based on this clinical study and conditions. Survival of patients in the high range of IL-2 expression was 18 months, as compared to 14 months for patients in the low range of tumoral TGF-β2 expression.

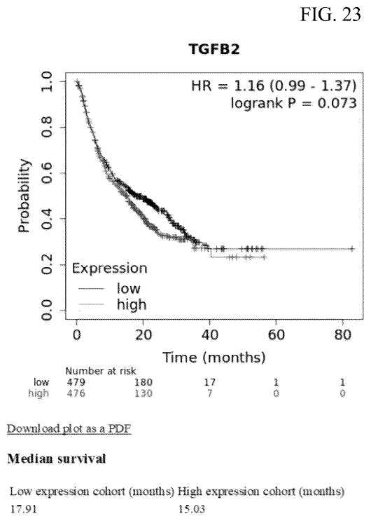

FIG. 23 shows that for use of a checkpoint inhibitor, a low level of TGF-β2 slightly increased survival. Survival of patients in the low range of TGF-β2 expression was 18 months, as compared to 15 months for patients in the high range of TGF-β2 expression.

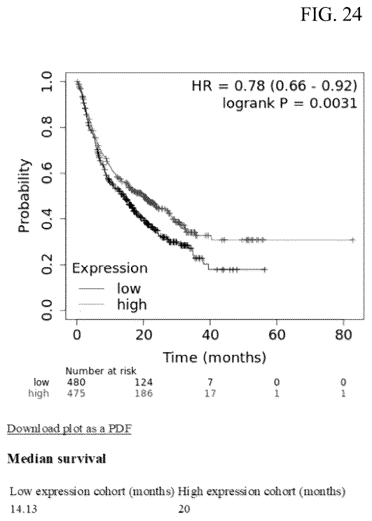

Importantly, FIG. 24 shows that for use of a checkpoint inhibitor, a low level of TGF-β2 along with a high level of IL-2 significantly increased survival. FIG. 24 shows overall survival based on stratification of the ratio IL2/TGFB2 expressions. Logrank P value in FIG. 24 indicates high significance for improved survival using a therapeutic combination of an antisense TGF-β2 inhibitor, an IL-2 immunotherapy agent, and a checkpoint inhibitor based on this clinical study and conditions. Survival of patients in the high range of IL2/TGFB2 expressions was 20 months, as compared to only 14 months for patients in the low range of IL2/TGFB2 expressions. This clinical data showed a synergistic effect of the therapeutic combination of an antisense TGF-β2 inhibitor, an IL-2 immunotherapy agent, and a checkpoint inhibitor.

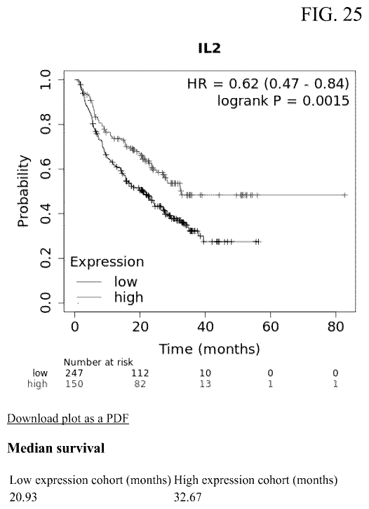

The synergistic effect of the therapeutic combination of an antisense TGF-β2 inhibitor, an IL-2 immunotherapy agent, and a checkpoint inhibitor was found to be markedly increased for melanoma individually. For the 423 melanoma patients, FIG. 25 shows that for use of a checkpoint inhibitor, a high level of IL-2 significantly increased survival. Survival of patients in the high range of IL-2 expression was 33 months, as compared to 21 months for patients in the low range of IL-2 expression.

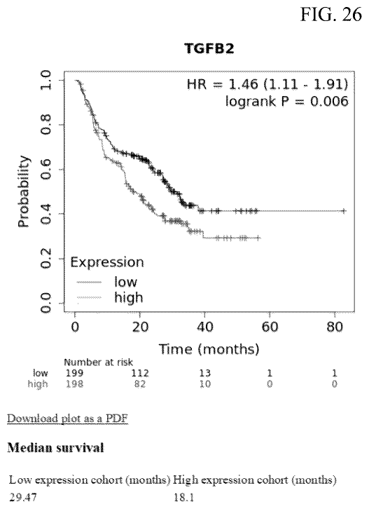

For the 423 melanoma patients, FIG. 26 shows that for use of a checkpoint inhibitor, a low level of TGF-β2 significantly increased survival. Survival of patients in the low range of TGF-β2 expression was 29 months, as compared to 18 months for patients in the high range of TGF-β2 expression.

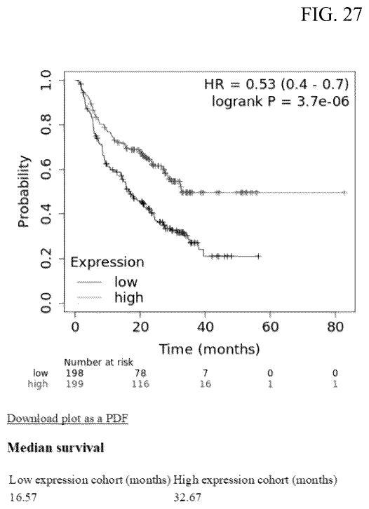

Importantly, FIG. 27 shows that in the 423 melanoma patients, for use of a checkpoint inhibitor, a low level of TGF-β2 along with a high level of IL-2 significantly increased survival. FIG. 24 shows overall survival based on stratification of the ratio IL2/TGFB2 expressions. Logrank P value in FIG. 24 indicates unexpectedly high significance for improved survival using a therapeutic combination of an antisense TGF-β2 inhibitor, an IL-2 immunotherapy agent, and a checkpoint inhibitor based on this clinical study and conditions. Survival of patients in the high range of IL2/TGFB2 expressions was 20 months, as compared to only 14 months for patients in the low range of IL2/TGFB2 expressions. This clinical data showed a synergistic effect of the therapeutic combination of an antisense TGF-β2 inhibitor, an IL-2 immunotherapy agent, and a checkpoint inhibitor.

The synergistic effect of the therapeutic combination of an antisense TGF-β2 inhibitor, an IL-2 immunotherapy agent, and a checkpoint inhibitor was found to be markedly increased for a PD-1 checkpoint inhibitor individually in cancer patients.

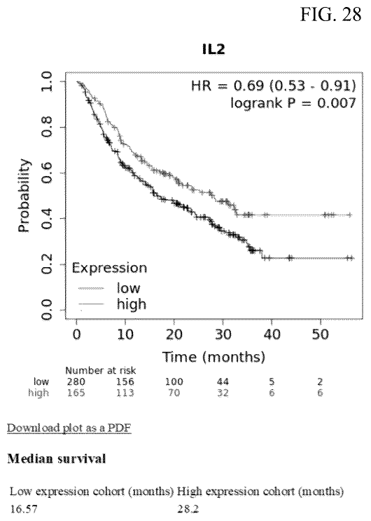

For all cancer patients, FIG. 28 shows that for use of a PD-1 checkpoint inhibitor, a high level of IL-2 significantly increased survival. Survival of patients in the high range of IL-2 expression was 28 months, as compared to 17 months for patients in the low range of IL-2 expression.

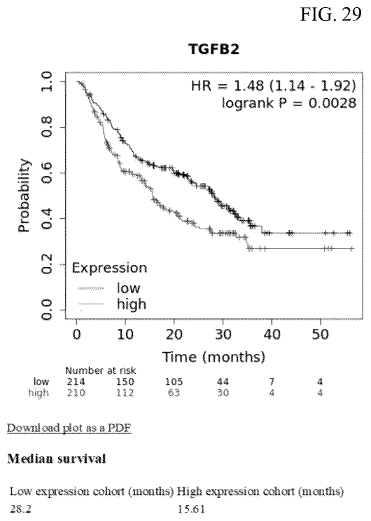

For cancer patients, FIG. 29 shows that for use of a PD-1 checkpoint inhibitor, a low level of TGF-β2 significantly increased survival. Survival of patients in the low range of TGF-β2 expression was 28 months, as compared to 16 months for patients in the high range of TGF-β2 expression.

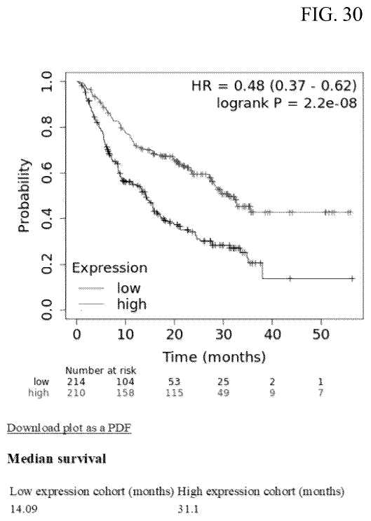

Importantly, FIG. 30 shows that in cancer patients, for use of a PD-1 checkpoint inhibitor, a low level of TGF-β2 along with a high level of IL-2 significantly increased survival. FIG. 30 shows overall survival based on stratification of the ratio IL2/TGFB2 expressions. Logrank P value in FIG. 30 indicates unexpectedly high significance for improved survival using a therapeutic combination of an antisense TGF-β2 inhibitor, an IL-2 immunotherapy agent, and a PD-1 checkpoint inhibitor based on this clinical study and conditions. Survival of patients in the high range of IL2/TGFB2 expressions was 31 months, as compared to 14 months for patients in the low range of IL2/TGFB2 expressions. This clinical data showed a synergistic effect of the therapeutic combination of an antisense TGF-β2 inhibitor, an IL-2 immunotherapy agent, and a PD-1 checkpoint inhibitor.

Example 6. A clinical study was performed to understand overall survival of cancer patients with a therapeutic combination of an antisense TGF-β2 inhibitor, an IL-2 immunotherapy agent, and a checkpoint inhibitor. (A clinical study was performed for understanding overall survival of melanoma treated with immunotherapy (N=423) (See KM plotter for immunotherapy, available at https://kmplot.com/analysis/index.php?p=service&cancer=immunotherapy) and pancreatic cancer (N=177) (KM Plotter Pan-cancer RNA-seq, https://kmplot.com/analysis/index.php?p=service&cancer=pancancer_rnaseq).

This clinical study showed that overall survival with a therapeutic combination of an antisense TGF-β2 inhibitor, an IL-2 immunotherapy agent, and a checkpoint inhibitor was not improved when the checkpoint inhibitor was CTLA-4 or PD-L1. Thus, in other examples herein the improvement in overall survival with a therapeutic combination of an antisense TGF-β2 inhibitor, an IL-2 immunotherapy agent, and a PD-1 checkpoint inhibitor was surprisingly advantageous. Details of the data are set forth below.

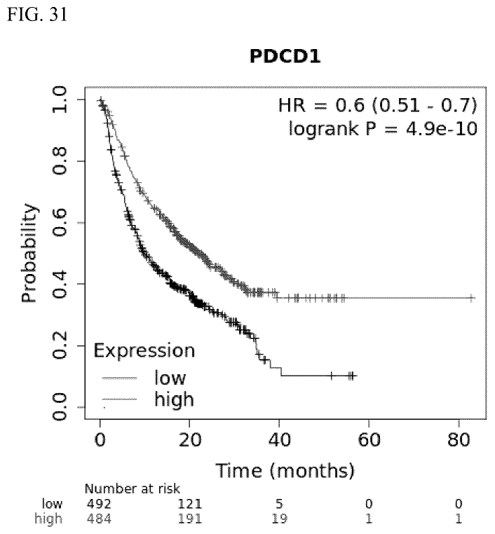

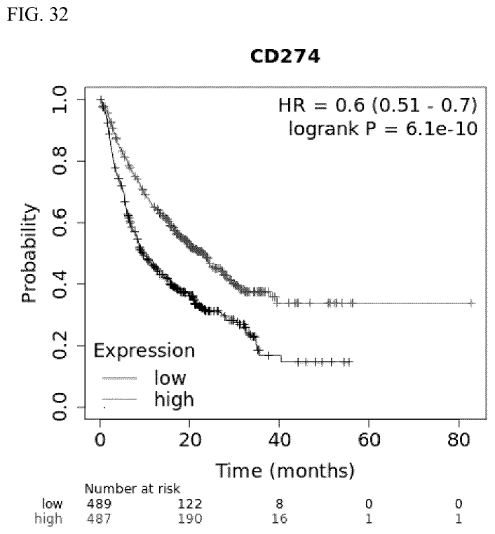

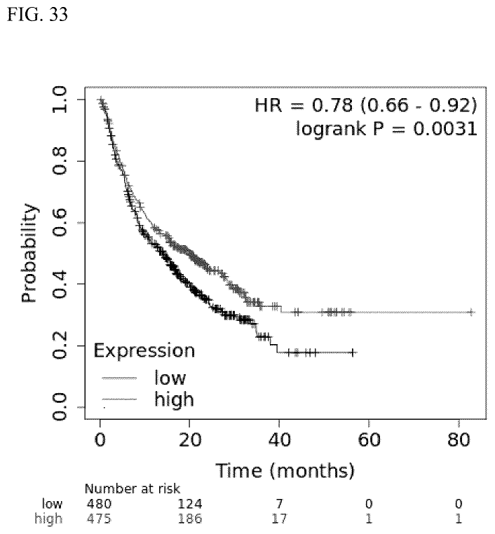

FIGS. 31-33 show results for three stratifying biomarkers, namely PDCD1, CD274, and the ratio IL-2/TGF-β2, for any checkpoint inhibitor (PD-1, CTLA-4 or PD-L1) and all tumor types (N=976). Each biomarker showed improved overall survival at high expression levels.

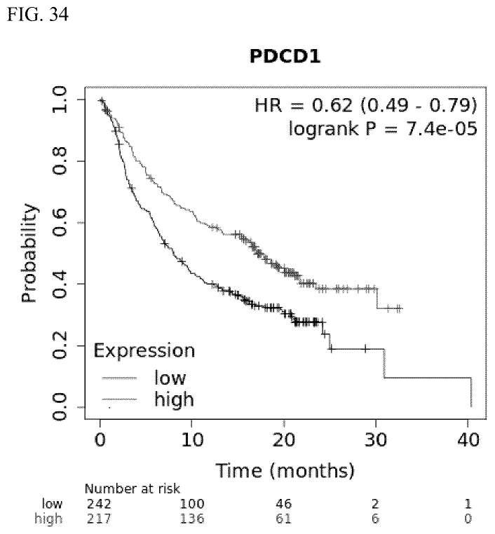

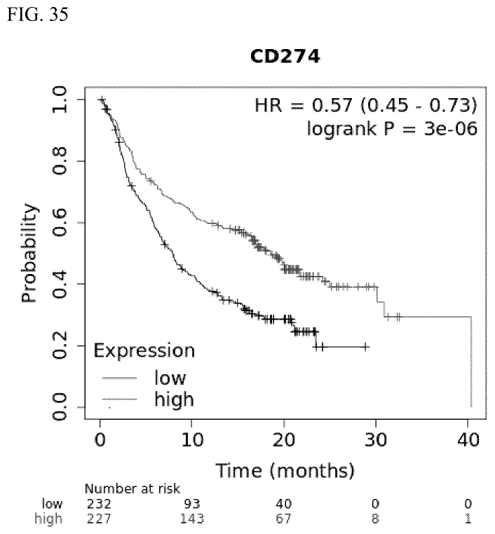

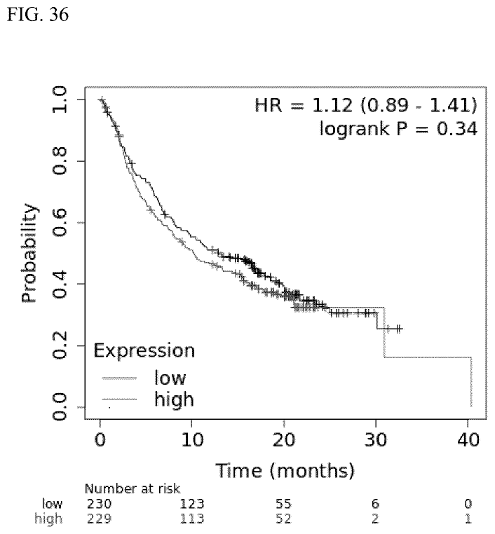

FIGS. 34-36 show results for three stratifying biomarkers, namely PDCD1, CD274, and the ratio IL-2/TGF-β2, for PD-L1 checkpoint inhibitor and all tumor types (N=955). Each biomarker except IL-2/TGF-β2 showed improved overall survival at high expression levels. Thus, PD-L1 was not a preferred checkpoint inhibitor.

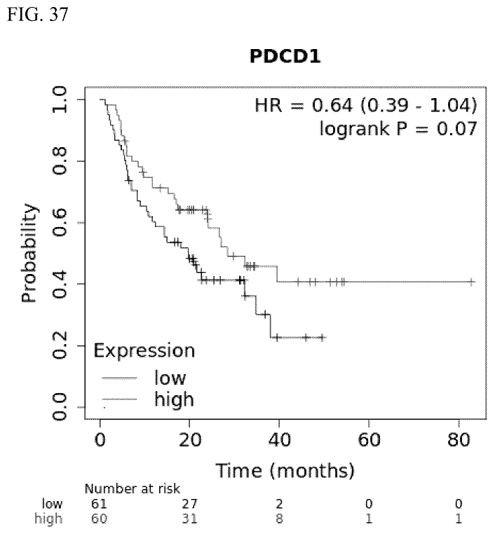

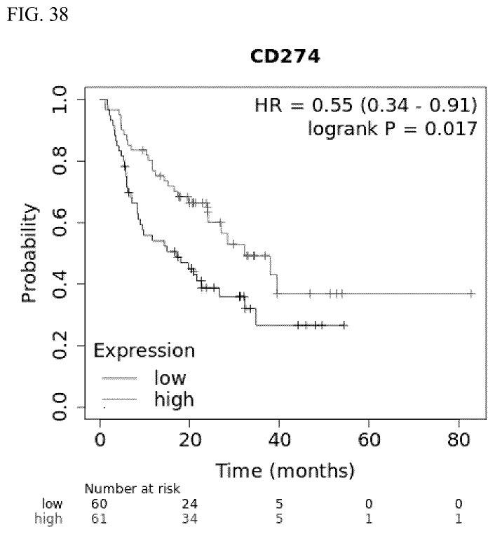

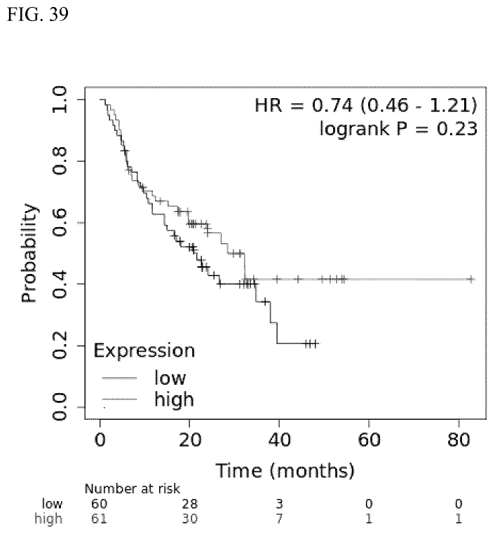

FIGS. 37-39 show results for three stratifying biomarkers, namely PDCD1, CD274, and the ratio IL-2/TGF-β2, for CTLA-4 checkpoint inhibitor and all tumor types (N=121). Only CD274 biomarker showed slightly improved overall survival at high expression levels. Thus, CTLA-4 was not a preferred checkpoint inhibitor.

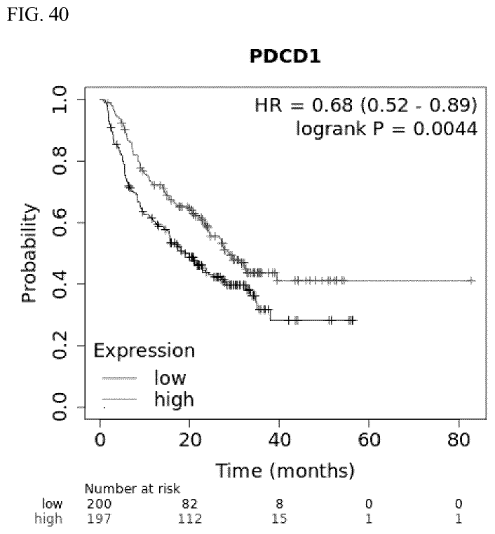

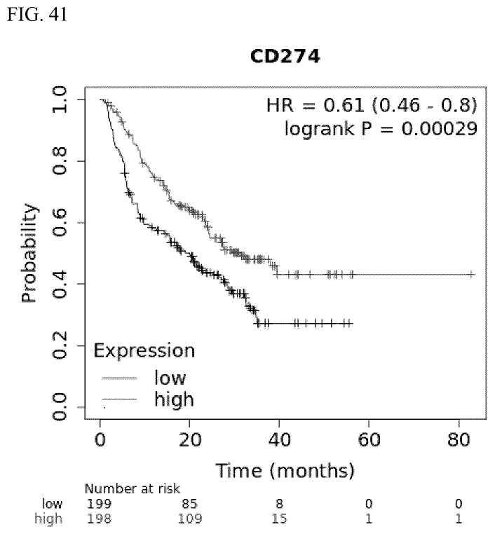

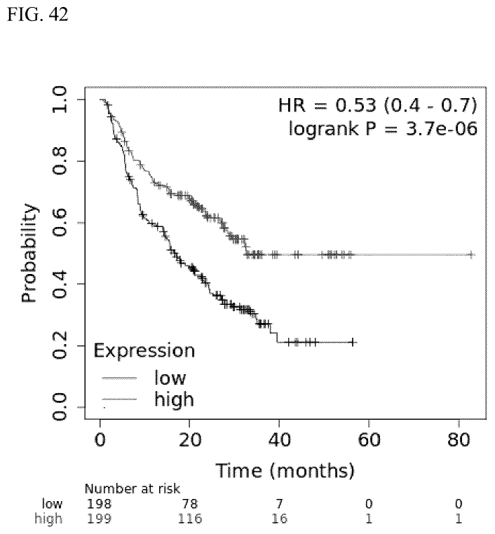

FIGS. 40-42 show results for three stratifying biomarkers, namely PDCD1, CD274, and the ratio IL-2/TGF-β2, for any checkpoint inhibitor with melanoma patients (N=397). Each biomarker showed improved overall survival at high expression levels.

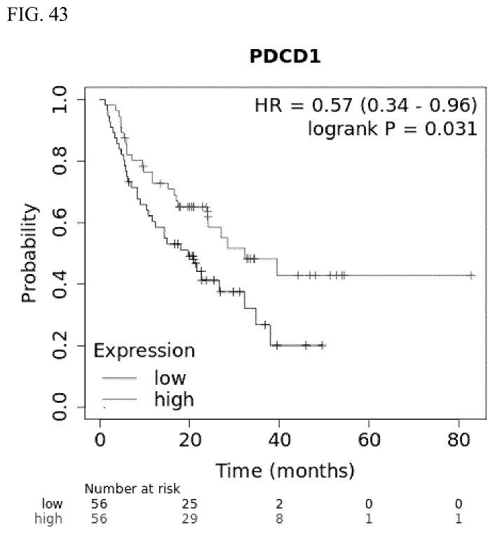

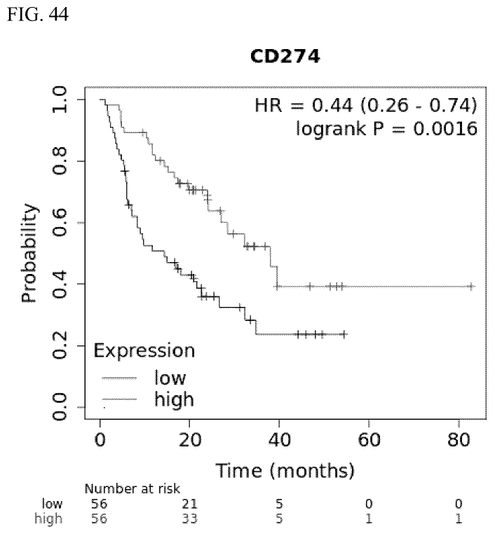

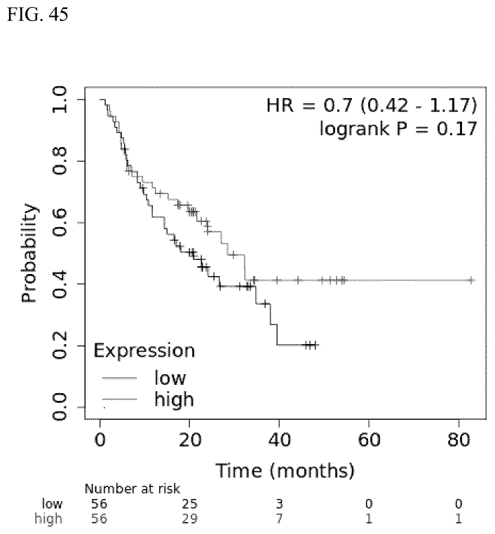

FIGS. 43-45 show results for three stratifying biomarkers, namely PDCD1, CD274, and the ratio IL-2/TGF-β2, for CTLA-4 checkpoint inhibitor with melanoma patients (N=112). Each biomarker except IL-2/TGF-β2 showed slightly improved overall survival at high expression levels. Thus, CTLA-4 was not a preferred checkpoint inhibitor.

What is claimed is:

1. An antisense agent for inhibiting or suppressing expression of TGF-β2 in combination with a checkpoint inhibitor agent for use in treating or ameliorating the symptoms of cancer in a human subject or animal.

2. Use of an antisense agent for inhibiting or suppressing expression of TGF-β2 in the preparation of a medicament for treating or ameliorating the symptoms of a cancer in a human subject or animal in combination with a checkpoint inhibitor agent.

3. A method for treating or ameliorating the symptoms of cancer in a human or animal subject in need, the method comprising:
administering a therapeutically effective amount of an antisense agent for inhibiting or suppressing expression of TGF-β2 to the subject;
administering a therapeutically effective amount of a checkpoint inhibitor agent to the subject.

4. The agent of claim 1, in combination with an interleukin immunotherapeutic agent.

5. The use of claim 2, in combination with an interleukin immunotherapeutic agent.

6. The method of claim 3, comprising administering a therapeutically effective amount of an interleukin immunotherapeutic agent to the subject.

7. The agent, use or method of any of claims 1-6, wherein the agent for inhibiting or suppressing expression of TGF-β2, the checkpoint inhibitor, and the interleukin immunotherapeutic agent are administered concurrently, simultaneously, sequentially, or separately in time.

8. The agent, use or method of any of claims 1-6, wherein the agent for inhibiting or suppressing expression of TGF-β2, the checkpoint inhibitor, and the interleukin immunotherapeutic agent are administered separately or in combined formulation by injection or infusion.

9. The agent, use or method of any of claims 1-6, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

10. The agent, use or method of any of claims 1-6, wherein the agent for inhibiting or suppressing expression of TGF-β2 is a TGF-β2-specific antisense oligonucleotide complementary to a TGF-β2 transcript and 15-30 nucleotides in length.

11. The agent, use or method of any of claims 1-6, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

12. The agent, use or method of any of claims 1-6, wherein the agent for inhibiting or suppressing expression of TGF-β2 is one or more TGF-β2-specific antisense oligonucleotides as shown below (Table 1), complementary to a TGF-β2 transcript:

| SEQ ID | | |
|---|---|---|
| NO: | Ref. | ANTISENSE SEQUENCE |
| 1 | — | GTTCGTTTAG AGAACAGATC |
| 2 | — | TAAAGTTCGT TTAGAGAACA G |
| 3 | — | AGCCCTGTAT ACGAC |
| 4 | — | GTAGGTAAAA ACCTAATAT |
| 5 | — | CGTTTAGAGA ACAGATCTAC |
| 6 | — | CATTGTAGAT GTCAAAAGCC |
| 7 | — | CTCCCTCATG GTGGCAGTTG A |
| 8 | — | CGGCATGTCT ATTTTGTA |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

and chemically-modified variants thereof, LNA variants thereof, gapmer variants thereof, and any combination or pooling thereof.

13. The agent, use or method of claim 12, wherein the TGF-β2-specific antisense oligonucleotides have no more than one or two mismatches as compared to a target human TGF-β2.

14. The agent, use or method of claim 12, wherein the TGF-β2-specific antisense oligonucleotides reduce a TGF-β2 transcript level by at least 60%, or at least 70%, or at least 80%, or at least 90%.

15. The agent, use or method of claim 12, wherein the TGF-β2-specific antisense oligonucleotides reduce any TGF-β1 transcript level and any TGF-β3 transcript level by less than 10%, or less than 5%, or less than 1%.

16. The agent, use or method of claim 12, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

17. The agent, use or method of claim 12, wherein the antisense agent is conjugated to a polyethylene glycol, a lipid, or a triantenarry N-acteyl-galactosamine.

18. The agent, use or method of any of claims 1-6, wherein each agent comprises a carrier of sterile water for injection, saline, isotonic saline, or a combination thereof, which may be the same or different for each agent.

19. The agent, use or method of any of claims 1-6, wherein the agents are substantially free of excipients.

20. The agent, use or method of any of claims 1-6, wherein the agents are stable in a carrier substantially free of excipients for at least 14 days at 37° C.

21. The agent, use or method of any of claims 1-6, wherein the checkpoint inhibitor agent is an inhibitor of PD-1.

22. The agent, use or method of any of claims 1-6, wherein the checkpoint inhibitor agent is pembrolizumab, nivolumab, cemiplimab, spartalizumab, atezolizumab, avelumab, or durvalumab.

23. The agent, use or method of any of claims 1-6, wherein the interleukin immunotherapeutic agent is a natural IL-2, a high dose IL-2, a recombinant IL-2, or aldesleukin.

24. The agent, use or method of any of claims 1-6, comprising selecting subjects who benefit from the agent, use or method based on levels of one or more biomarkers TGF-β2, IL-2, CD19, IRF5, ITGAM, and a combination thereof.

25. The agent, use or method of claim 24, wherein the one or more biomarkers is IRF5 and the subject is selected when expression of IRF5 is at a level above the median.

26. The agent, use or method of claim 24, wherein the one or more biomarkers is ITGAM and the subject is selected when expression of ITGAM is at a level above the median.

27. The agent, use or method of any of claims 1-6, wherein the subject after the administration or use has a decreased level of TGF-β2 as compared to before the administration or use.

28. The agent, use or method of any of claims 1-6, wherein the subject after the administration or use has an increased level of IRF5 as compared to before the administration or use.

29. The agent, use or method of any of claims 1-6, wherein the subject after the administration or use has a decreased level of ITGAM as compared to before the administration or use.

30. The agent, use or method of any of claims 1-6, comprising administering a therapeutically effective amount of an expression product of IRF5 or ITGAM to the subject.

31. The agent, use or method of claim 30, wherein the expression product is an mRNA, polypeptide, protein, or fragment thereof, or combination thereof.

32. The agent, use or method of any of claims 1-6, wherein the administration or use decreases mortality rate at month 6, 12, 18, 24, 30, or 36.

33. The agent, use or method of any of claims 1-6, wherein the administration or use increases overall survival rate at month 6, 12, 18, 24, 30, or 36.

34. The agent, use or method of any of claims 1-6, in combination with any one or more medicaments comprising a targeted cancer drug, a cancer growth blocker, an EGFR inhibitor, and combinations thereof.

35. The agent, use or method of any of claims 1-6, in combination with any one or more medicaments selected from bevacizumab, everolimus, belzutifan, dabrafenib, trametinib, and combinations thereof.

36. The agent, use or method of any of claims 1-6, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

37. The agent, use or method of any of claims 1-6, in combination with any one or more medicaments which are EGFR inhibitors selected from erlotinib, gefitinib, afatinib, osimertinib, dacomitininb, and combinations thereof.

38. The agent, use or method of any of claims 1-6, in combination with a chemotherapy medicament.

39. The agent, use or method of any of claims 1-6, in combination with radiation therapy or electric field therapy.

40. An antisense agent for inhibiting or suppressing expression of TGF-β2 in combination with an interleukin immunotherapeutic agent for use in treating or ameliorating the symptoms of cancer in a human subject or animal.

41. Use of an antisense agent for inhibiting or suppressing expression of TGF-β2 in the preparation of a medicament for treating or ameliorating the symptoms of a cancer in a human subject or animal in combination with an interleukin immunotherapeutic agent.

42. A method for treating or ameliorating the symptoms of cancer in a human or animal subject in need, the method comprising:
administering a therapeutically effective amount of an antisense agent for inhibiting or suppressing expression of TGF-β2 to the subject;
administering a therapeutically effective amount of an interleukin immunotherapeutic agent to the subject.

43. The agent, use or method of any of claims 40-42, wherein the agent for inhibiting or suppressing expression of TGF-β2, and the interleukin immunotherapeutic agent are administered concurrently, simultaneously, sequentially, or separately in time.

44. The agent, use or method of any of claims 40-42, wherein the agent for inhibiting or suppressing expression of TGF-β2, and the interleukin immunotherapeutic agent are administered separately or in combined formulation by injection or infusion.

45. The agent, use or method of any of claims 40-42, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, or a multiple myeloma.

46. The agent, use or method of any of claims 40-42, wherein the agent for inhibiting or suppressing expression of TGF-β2 is a TGF-β2-specific antisense oligonucleotide complementary to a TGF-β2 transcript and 15-30 nucleotides in length.

47. The agent, use or method of any of claims 40-42, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

48. The agent, use or method of any of claims 40-42, wherein the agent for inhibiting or suppressing expression of TGF-β2 is one or more TGF-β2-specific antisense oligonucleotides as shown in Table 1, complementary to a TGF-β2 transcript.

49. The agent, use or method of claim 48, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

50. The agent, use or method of claim 48, wherein the antisense agent is conjugated to a polyethylene glycol, a lipid, or a triantenarry N-acteyl-galactosamine.

51. The agent, use or method of any of claims 40-42, wherein each agent comprises a carrier of sterile water for injection, saline, isotonic saline, or a combination thereof, which may be the same or different for each agent.

52. The agent, use or method of any of claims 40-42, wherein the agents are substantially free of excipients.

53. The agent, use or method of any of claims 40-42, wherein the agents are stable in a carrier substantially free of excipients for at least 14 days at 37° C.

54. The agent, use or method of any of claims 40-42, wherein the interleukin immunotherapeutic agent is a natural IL-2, a high dose IL-2, a recombinant IL-2, or aldesleukin.

55. The agent, use or method of any of claims 40-42, wherein the administration or use decreases mortality rate at month 6, 12, 18, 24, 30, or 36.

56. The agent, use or method of any of claims 40-42, wherein the administration or use increases survival rate at month 6, 12, 18, 24, 30, or 36.

57. The agent, use or method of any of claims 40-42, in combination with any one or more medicaments comprising a targeted cancer drug, a cancer growth blocker, an EGFR inhibitor, and combinations thereof.

58. The agent, use or method of any of claims 40-42, in combination with any one or more medicaments selected from bevacizumab, everolimus, belzutifan, dabrafenib, trametinib, and combinations thereof.

59. The agent, use or method of any of claims 40-42, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

60. The agent, use or method of any of claims 40-42, in combination with any one or more medicaments which are EGFR inhibitors selected from erlotinib, gefitinib, afatinib, osimertinib, dacomitininb, and combinations thereof.

61. The agent, use or method of any of claims 40-42, in combination with a chemotherapy medicament.

62. The agent, use or method of any of claims 40-42, in combination with radiation therapy or electric field therapy.

63. The agent, use or method of any of claims 40-42, comprising selecting subjects who benefit from the agent, use or method based on levels of one or more biomarkers TGF-β2, IRF5, ITGAM, and a combination thereof.

64. The agent, use or method of any of claims 40-42, comprising selecting subjects who benefit from the agent, use or method based on levels of one or more biomarkers TGF-β2, IRF5, ITGAM, neoantigen, mutational load, macrophage and a combination thereof.

65. The agent, use or method of claim 64, wherein the one or more biomarkers is IRF5 and the subject is selected when expression of IRF5 is at a level below the median.

66. The agent, use or method of claim 64, wherein the one or more biomarkers is tumor associated macrophage and the subject is selected when tumor associated macrophage is below average.

67. The agent, use or method of claim 64, wherein the one or more biomarkers is tumor neoantigen mutation load and the subject is selected when neoantigen tumor load is below average.

68. The agent, use or method of any of claims 40-42, comprising administering a therapeutically effective amount of an agent for inhibiting or suppressing expression of ITGAM or IRF5 to the subject.

69. The agent, use or method of claim 68, wherein the agent for inhibiting or suppressing expression of ITGAM or IRF5 is an antisense oligonucleotide targeted to ITGAM or IRF5, respectively.

70. A kit for treating or ameliorating the symptoms of cancer, the kit comprising:
a therapeutically effective amount of an antisense agent for inhibiting or suppressing expression of TGF-β2; and
a therapeutically effective amount of a checkpoint inhibitor agent.

71. The kit of claim 70, comprising a therapeutically effective amount of an interleukin immunotherapeutic agent.

72. The kit of claim 70, wherein the cancer is a pancreatic cancer, a melanoma, a skin cancer, a lung cancer, a breast cancer, a prostate cancer, a colorectal cancer, a kidney cancer, a stomach cancer, an ovarian cancer, a cervical cancer, a liver cancer, a thymus cancer, or a multiple myeloma.

73. The kit of claim 70, wherein the agent for inhibiting or suppressing expression of TGF-β2 is a TGF-β2-specific antisense oligonucleotide complementary to a TGF-β2 transcript and 15-30 nucleotides in length.

74. The kit of claim 70, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

75. The kit of claim 70, wherein the agent for inhibiting or suppressing expression of TGF-β2 is one or more TGF-β2-specific antisense oligonucleotides as shown in Table 1, complementary to a TGF-β2 transcript.

76. The kit of claim 70, wherein the TGF-β2-specific antisense oligonucleotides have no more than one or two mismatches as compared to a target human TGF-β2.

77. The kit of claim 70, wherein the TGF-β2-specific antisense oligonucleotides reduce a TGF-β2 transcript level by at least 60%, or at least 70%, or at least 80%, or at least 90%.

78. The kit of claim 70, wherein the TGF-β2-specific antisense oligonucleotides reduce any TGF-β1 transcript level and any TGF-β3 transcript level by less than 10%, or less than 5%, or less than 1%.

79. The kit of claim 70, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

80. The kit of claim 70, wherein the antisense agent is conjugated to a polyethylene glycol, a lipid, or a triantenarry N-acteyl-galactosamine.

81. The kit of claim 70, wherein each agent comprises a carrier of sterile water for injection, saline, isotonic saline, or a combination thereof, which may be the same or different for each agent.

82. The kit of claim 70, wherein the agents are substantially free of excipients.

83. The kit of claim 70, wherein the agents are stable in a carrier substantially free of excipients for at least 14 days at 37° C.
